(19) United States
(12) Patent Application Publication
(10) Pub. No.: US 2023/0310609 A1
(43) Pub. Date: Oct. 5, 2023

(54) INJECTABLE HYDROGELS AND METHODS OF CAPTURING CELLS USING THE SAME

(71) Applicant: VIRGINIA POLYTECHNIC INSTITUTE AND STATE UNIVERSITY, Blacksburg, VA (US)

(72) Inventors: Zerin Mahzabin Khan, Blacksburg, VA (US); Scott S. Verbridge, Blacksburg, VA (US); Timothy E. Long, Blacksburg, VA (US); Eli Vlaisavlijevich, Blacksburg, VA (US)

(21) Appl. No.: 18/021,637

(22) PCT Filed: Aug. 21, 2021
(86) PCT No.: PCT/US2021/047033

§ 371 Date: Feb. 16, 2023

Related U.S. Application Data
(60) Provisional application No. 63/068,961, filed on Aug. 21, 2020.

(51) Int. Cl. A61K 41/00; A61K 9/00; A61K 9/06; A61P 35/00; A61K 47/34; A61L 27/52; A61L 27/54; A61L 27/26

(52) U.S. Cl. CPC A61K 41/0033; A61K 9/0024; A61K 9/06; A61P 35/00; A61K 47/34; A61L 27/52; A61L 27/54; A61L 27/26; A61L 2400/06; A61B 2017/320069

(57) ABSTRACT

Described in several embodiments herein are injectable hydrogels that are capable of attracting one or more cells, in situ. In some embodiments, the cells are cancer cells, such as cancer stem cells. Also described herein are methods of using the injectable hydrogels to fill a cavity in a subject. Also described herein are methods of treating a cancer by injecting an injectable hydrogel in a cavity in a subject formed from resecting a tumor and applying an external stimulus to the injected injectable hydrogel or area proximate to the injected injectable hydrogel.

### CROSS-REFERENCE TO RELATED APPLICATIONS

This application claims the benefit of and priority to co-pending U.S. Provisional Patent Application No. 63/068,961, filed on Aug. 21, 2020 entitled “INJECTABLE HYDROGELS AND METHODS OF CAPTURING CELLS USING THE SAME,” the contents of which is incorporated by reference herein in its entirety.

### STATEMENT REGARDING FEDERALLY SPONSORED RESEARCH

This invention was made with government support under Grant No. CBET-1652112 awarded by the National Science Foundation. The government has certain rights in the invention.

### TECHNICAL FIELD

The subject matter disclosed herein is generally directed to injectable hydrogels and their methods of use, particularly to capture cells.

### BACKGROUND

Glioblastoma multiforme (GBM) is the most aggressive type of primary brain tumor in adults. Its infiltrative capacity allows GBM to utilize a variety of strategies to diffuse to surrounding brain tissues. Current treatments employ “search and destroy” strategies such as surgery, chemotherapy, and radiotherapy to eradicate GBM. However, the glioma cancer stem cell (GSC) population resistant to therapy remain, which leads to tumor recurrences and the poor prognosis of the disease. As such, there exists a need for improved treatments for GBM and other cancers, such as those where cancer stem cells play roles in continuation of the disease. Citation or identification of any document in this application is not an admission that such a document is available as prior art to the present invention.

### SUMMARY

Described in several example embodiments herein are injectable hydrogels comprising:
a hydrogel matrix comprising one or more polymers;
a wt % of water that is any non-zero wt % that ranges from about 0 up to, but not including, 75 wt %;
optionally, one or more agents, wherein one of the one or more agents is optionally a cell migration modulator,
wherein the storage modulus of the injectable hydrogel ranges from about 3 to about 100 kPa, from about 3 to about 50 kPa, or from about 3 to about 25 kPa.

In certain example embodiments, the wt % of water is any non-zero wt % ranging from about 0 to about 50 wt %, 0 to about 25 wt %, about 25 wt % to about 50 wt %, about 25 wt % up to, but not including 75 wt %, or about 50 wt % up to, but not including, 75 wt %.

In certain example embodiments, wherein the injectable hydrogel has a storage modulus effective for implantation into brain tissue.

In certain example embodiments, the injectable hydrogel is biocompatible.

In certain example embodiments, the injectable hydrogel is responsive to a stimulus.

In certain example embodiments, the stimulus is an abiotic environmental condition, a chemical, a biologic agent, an energy, or any combination thereof.

In certain example embodiments, injectable hydrogel is an agent eluting hydrogel and is capable of releasing one or more agents into the environment surrounding the hydrogel.

In certain example embodiments, the stimulus is capable of triggering agent elution from the hydrogel, agent activation, agent deactivation, or any combination thereof.

In certain example embodiments, the cell migration modulator is a cell attractant.

In certain example embodiments, the cell attractant is
a. a cancer stem cell attractant;
b. a circulating cancer cell attractant;
c. a migrating cancer cell attractant;
d. a disseminating cancer cell attractant;
e. a glioma cell attractant;
f. a tumor microenvironment cell attractant;
g. an immune cell attractant;
h. a cancer cell attractant;
i. a cancer-associated fibroblast attractant;
j. a tumor initiating cell attractant; or
k. any combination thereof.

In certain example embodiments, the one or more polymers comprises a polymer having one or more hydrophilic groups.

In certain example embodiments, each of the one or more hydrophilic groups individually selected from the group consisting of: —NH₂, —COOH, —OH, —CONH₂, —CONH—, and —SO₃H.

In certain example embodiments, the injectable hydrogel is cationic, nonionic, or anionic.

In certain example embodiments, the one or more polymers comprises a natural polymer, a synthetic polymer, or a combination thereof.

In certain example embodiments, the one or more polymers are chemically crosslinked, physically crosslinked, or both.

In certain example embodiments, the one or more polymers are each individually selected from polyethylene glycol (PEG), chitosan, Poly(-hydroxyethyl methacrylate) (PHEMA), 2-Hydroxyethyl methacrylate (HEMA), Hydroxyethoxyethyl metha-crylate (HEEMA), Hydroxydiethoxyethylmethacrylate (HDEEMA), Methoxyethyl methacrylate (MEMA), Methoxyethoxyethyl methacrylate (MEEMA), Methoxy-diethoxyethyl methacrylate (MDEEMA), Ethylene glycol dimethacrylate (EGDMA), N-vinyl-2-pyrrolidone (NVP), N-isopropyl AAm (NIPAAm), Vinyl acetate (VAc), Acrylic acid (AA), N-(2-hydroxypropyl) methacrylamide (HPMA), Ethylene glycol (EG), PEG acrylate (PEGA), PEG methacrylate (PEGMA), PEG diacrylate (PEGDA), PEG dimethacrylate (PEGDMA), Methacrylic acid (MAA), PEG-PEGMA, Carboxymethyl cellulose (CMC), Polyvinylpyrrolidone (PVP), an Acrylamide/acrylic acid copolymer, linear cationic polyallylammonium chloride, Poly(N-isopropyl acrylamide) (PNIPAM), self-assembling peptides, acrylate-modified PEG and acrylate-modified hyaluronic acid, heparin, amine end-functionalized 4-arm star-PEG, or any combination thereof.

In certain example embodiments, at least one of the one or more polymers is PEGDA.

In certain example embodiments, the injectable hydrogel is a thiol-Michael addition hydrogel.

In certain example embodiments, the injectable hydrogel is a reaction product of a polymer comprising at least one Michael acceptor and a thiol compound reacted in the presence of an aqueous base.

In certain example embodiments, the one or more polymers comprises a monomer that is a Michael acceptor.

In certain example embodiments, the Michael acceptor is acrylate, vinyl nitrile, vinyl nitro, vinyl phosphonate, vinyl sulfonate, or a compound comprising an enone.

In certain example embodiments, the thiol compound is a multi-arm, thiol terminated polymer comprising a backbone consisting of: poly(ethylene glycol), polycaprolactam, poly(propylene glycol), and poly(lactide) chains, and any water-soluble polysaccharide functionalized with 3 or more thiol groups per chain.

In certain example embodiments, the thiol compound is a multi-arm, thiol-terminated polyethylene glycol (PEG) oligomer or ethoxylated trimethylolpropane tri-3-mercaptopropionate.

In certain example embodiments, the thiol-terminated PEG oligomer has an average molecular weight less than about 100,000 g/mol.

In certain example embodiments, the aqueous base is an inorganic carbonate, an inorganic bicarbonate, a buffer having a pH ranging from 7.4-14, an amine base, or any combination thereof.

In certain example embodiments, the aqueous base is NaHCO₃.

In certain example embodiments, the concentration of the aqueous base is 0.1 M-0.25 M.

In certain example embodiments, the injectable hydrogel is capable of capturing and/or retaining one or more cells.

In certain example embodiments, one or more of the one or more agents is selected from the group consisting of: DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, agent sensitizers, imaging agents, chemotherapeutic agents, chemokines, anti-migratory compounds capable of inhibit inhibiting chemokine receptors to decrease cell invasion, and any combination thereof.

Described in certain example embodiments herein are methods of attracting cells to and capturing cells an injectable hydrogel as described herein that is within a subject, the method comprising:
optionally releasing the optional cell migratory modulating agent from the injectable hydrogel so as to form a chemotaxis gradient in the environment around the hydrogel; and
applying a first external stimulus to the injectable hydrogel and/or body cavity of the subject, wherein the first external stimulus effective to stimulate migration of one or more cells within the subject to the injected injectable hydrogel; and
capturing and/or retaining one or more of the one or more cells within the hydrogel for a period of time.

In certain example embodiments, the one or more cells are selected from: cancer cells, cancer stem cells, circulating cancer cells, residual cancer cells, tumor microenvironment cells, immune cells, tumor initiating cells, cancer-associated fibroblasts, or any combination thereof.

In certain example embodiments, the method further comprises injecting, into a body cavity of a subject,
a. an injectable hydrogel of as described herein, or
b. one or more reagents capable of forming an injectable hydrogel as described herein so as to form an injected injectable hydrogel within the body cavity after injection.

In certain example embodiments, the method further comprises exposing the injected injectable hydrogel to a second external stimulus to the hydrogel after a period of time sufficient to capture and/or retain one or more cells in the injectable hydrogel.

In certain example embodiments, the second external stimulus is capable of modifying, modulating, inhibiting growth of, and/or killing one or more cells captured and/or retained in the injectable hydrogel.

In certain example embodiments, the second external stimulus is an energy and wherein the energy is an electric energy, a light energy, a magnetic energy, a thermal energy, an acoustic energy, a chemical energy, a biochemical energy, a radiation energy, or any combination thereof.

In certain example embodiments, the second external stimulus is an acoustic energy or an electric energy.

In certain example embodiments, the acoustic energy is ultrasound.

In certain example embodiments, the electric energy is delivered by one or more probes, wherein
a. a cathode probe or cathode end of a probe is positioned in, adjacent to, or in proximity to the injectable hydrogel; or
b. an anode probe or anode end of a probe is positioned in, adjacent to, or in proximity to the injectable hydrogel.

In certain example embodiments, the one or more probes or other energy sources capable of delivering the second external stimulus are placed in operable proximity to the injected injectable hydrogel.

In certain example embodiments, one or more of the one or more cells originates from the body cavity microenvironment.

In certain example embodiments, the second external stimulus is capable of
a. damaging one or more cells attracted to, in immediate proximity of, contained in the injected injectable hydrogel, or any combination thereof;
b. killing or ablating one or more cells attracted to, in immediate proximity of, contained in the injected injectable hydrogel; or any combination thereof;
c. modifying the one or more cells attracted to, in immediate proximity of, contained in the injected injectable hydrogel, or any combination thereof, or
d. any combination thereof.

In certain example embodiments, the body cavity is a surgical cavity.

In certain example embodiments, the surgical cavity is formed from removing a tumor from the body of the subject.

In certain example embodiments, the body cavity is in the brain of the subject.

In certain example embodiments, the tumor is a glioma.

Described in certain example embodiments herein are methods treating a cancer in a subject, comprising performing the method of attracting cells to and capturing cells in an injectable hydrogel described herein.

In certain example embodiments, the cancer is a glioblastoma.

In certain example embodiments, the glioblastoma is glioblastoma multiforme.

In certain example embodiments, the method further comprises imaging the injected injectable hydrogel after injecting into a subject.

In certain example embodiments, the method further comprises releasing one or more agents optionally included in the injectable hydrogel over a period of time.

In certain example embodiments, the method further comprises removing the injected hydrogel after one or more cells are collected therein.

The method of claim 46, further comprising delivering to the subject one or more chemotherapeutics, therapeutic radiation, or both. These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1B—Glioblastoma cell invasion and glioma cancer stem cell populations. FIG. 1A shows strategies employed by GBM to infiltrate into surrounding healthy brain tissue. See also Seano, G., & Jain, R. K. (2020). Angiogenesis, 23(1), 9-16. doi:10.1007/s10456-019-09691-z. FIG. 1B shows the cancer stem cell hypothesis where residual cancer stem cells drive tumor proliferation, self-renewal, and recurrence. See also Das, S., Srikanth, M. & Kessler, J. A. Cancer stem cells and glioma. Nature clinical practice. Neurology 4, 427-435, doi:10.1038/ncpneuro0862 (2008).

FIG. 2—Residual cancer cell entrapment into an injected injectable hydrogel. An exemplary embodiment is shown in which a glioblastoma tumor is removed surgically leaving a surgical cavity that contains, inter alia, residual cancer stem cells and glioblastoma cells. An injectable hydrogel of the present disclosure can be injected into the space where it forms to the surgical cavity. Residual cells are attracted to the injected injectable hydrogel by agent(s) present in the hydrogel and/or an external stimulus applied (such as an electrical or other energy). The cells captured and contained in the hydrogel can be killed or otherwise rendered inactive by agent(s) present in the hydrogel and/or an energy applied (e.g., ultrasound) to the hydrogel and/or removed by e.g., removal of the hydrogel from the cavity.

FIGS. 3A-3B—Thiol-Michael hydrogel synthesis. FIG. 3A shows a reaction scheme where NaHCO₃ base catalyzes the addition of a thiolate (trithiol thiocure) into an electron deficient alkene (polyethylene glycol diacrylate (PEGDA) with a 1:1 stochiometric ratio under aqueous conditions at a physiologic temperature. See also e.g., Moon, N. G., Pekkanen, A. M., Long, T. E., Showalter, T. N. & Libby, B. Thiol-Michael ‘click’ hydrogels as an imagable packing material for cancer therapy. Polymer 125, 66-75, doi:10.1016/j.polymer.2017.07.078 (2017). FIG. 3B shows an image of a hydrogel synthesized with 0.1 M NaHCO₃ and about 50 percent water.

FIGS. 4A-4B—Effect of hydration level and base concentration on hydrogel swelling. Hydrogels were swelled in phosphate buffer saline solution at 37 degrees C. in triplicate. FIG. 4A shows a graph demonstrating swelling kinetics obtained at 10-minute intervals for up to 180 minutes. FIG. 4B shows a graph demonstrating swelling ratios at equilibrium. The hydrogel formulation of 0.1M and 75% did not swell and reach equilibrium (indicated by x). 25 wt % hydrogels swelled the most. 50 wt % hydrogels swelled the least, has the least batch-to-batch variability (as evidenced by lowest standard deviation) and exhibited the best behavior for in vivo implantation. ** p-value <0.01, error bars represent the standard deviation.

FIGS. 5A-5B—Rheological properties of hydrogel formulations. All hydrogels were swelled for 12 hours in phosphate buffer saline solution at 37° C. before being subjected to the compression mode in an RSA-G2 at 2% shear strain and frequency sweep from 0.1 to 10 Hz at 37° C. Hydrogels in triplicate with dimensions of 10 mm×10 mm×2.5 mm were used for each formulation. Data reported at 1 Hz in the linear viscoelastic region. (FIG. 5A) Storage modulus. (FIG. 5B) Tan delta values for ratio of the loss modulus (G″) to the storage modulus (G′) approximating hydrogel stiffness. ** p-value <0.01, error bars represent standard deviations. 0.25M at 50 wt % possessed the highest storage modulus. The remaining formulations were within range corresponding to glioblastoma stiffening and normal brain tissue (e.g., 1-50 kPa) and the range at which cells become mechanoresponsive.

FIGS. 6A-6B—Hydrogel disintegration and stability under conditions mimicking cerebrospinal fluid exposure. Hydrogels were submerged in 14 mL of phosphate buffer saline solution containing calcium and magnesium ions in triplicate. (FIG. 6A) Disintegration kinetics. (FIG. 6B) Total mass loss after 15 days. ** p-value <0.01, error bars represent standard deviations. 0.25 M and 50 wt % hydrogels disintegrated the most quickly with the highest level of mass loss.

FIG. 7—Effect of hydrogel hydration level and base concentration on gel fraction. Each formulation was vacuum dried and submerged in dichloromethane to extract soluble components in triplicate. All 0.25 M hydrogels possessed gel fractions below 80% and therefore did not possess a sufficiently well crosslinked network. Differences among formulations not significant. 0.25 M gels were not evaluated further.

FIGS. 8A-8B—Cytotoxicity of hydrogels to normal human astrocytes (NHA). Hydrogels in quadruplet were pre-equilibrated with media before NHA were seeded on the surface at a density of 50,000 cells per hydrogel. Cell cultures were maintained at 37° C. with 5% CO2 in media. Cell viabilities were assessed with alamarBlue assay. (FIG. 8A) Cell viability over 7 days. (FIG. 8B) Cell viabilities on Day 1 vs Day 7. *** p-value <0.001 and **** p-value <0.0001, error bars represent standard deviations. All cell viabilities above 70% threshold for approval as implant by the FDA. 0.175 M at 50 wt % hydrogels demonstrated highest cell viabilities consistently over the course of 7 days. 25 wt % hydrogels had lowest cell viabilities at 7 days and were screened out from further study.

FIGS. 9A-9B—Immunogenicity and IL-6 secretion of normal human astrocytes upon exposure to hydrogels. NHA were seeded on the surface of hydrogels at a density of 50,000 cells per hydrogel and cultured for 15 days. In triplicate samples, the supernatant was collected and analyzed by ELISA to detect the concentration of IL-6 secretion. A scratch wound assay for NHA seeded on a 2D surface was performed as control on Day 5. (FIG. 9A) IL-6 secretion over 15 days. (FIG. 9B) Cumulative IL-6 secretion on Day 7, normalized to the NHA cell density. *** p-value <0.001, error bars represent standard deviations. IL-6 secretion peaks from Days 5-7 for both hydrogels and may help to promote invasion and migration of GBM cells and GSCs toward hydrogel. No significant difference in immunogenicity between the two remaining formulations.

FIG. 10—Morphology of normal human astrocytes exposed to hydrogel. NHA were seeded on surface at a density of 100,000 cells per hydrogel placed in PDMS molds for triplicate samples. Cell cultures were maintained for 15 days and imaged with confocal microscopy every other day for 15 days. Green (represented in greyscale) stain is GFAP, blue stain (represented in greyscale) is DAPI. Scale bar is 10 μm. Three separate fields of view were imaged per sample. Cells maintained a round morphology in hydrogels.

FIGS. 11A-11C—Astrocyte reactivity in response to hydrogels from confocal imaging. (FIG. 11A) Normalized GFAP expression of astrocytes seeded on hydrogels and 2D well plate condition. (FIG. 11B) NHA diameters for cells seeded on hydrogels. (FIG. 11C) Correlation between GFAP expression and NHA cell diameter upon exposure to hydrogels. * p-value <0.05, error bars represent standard deviations. NHA reactivity is higher in 0.1 M and 50 wt % hydrogel formulation. Astrogliosis corresponds to IL-6 secretion. For both hydrogels, astrocyte reactivity peaks during the first 5 days before stabilizing as cells adjust to environment. 0.175 M at 50 wt % is most suitable formulation for GBM application.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “administering” refers to any suitable administration for the agent(s) being delivered and/or subject receiving said agent(s) and can be oral, topical, intravenous, subcutaneous, transcutaneous, transdermal, intramuscular, intra joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracorneal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed. An agent can be synthetic or natural. An agent can be a chemical agent or biological agent, such as a molecule, compound, composition, system, or formulation.

As used herein, “biologic agent” refers to any compound, composition, molecule and the like that is made by a living organism and include, without limitation, polynucleotides (e.g. DNA, RNA), peptides and polypeptides, and chemical compounds (e.g. hormones, chemokines, and cytokines).

The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, glioblastoma, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof “A polymer” can be a three-dimensional network (e.g., the repeat units are linked together left and right, front and back, up and down), a two-dimensional network (e.g., the repeat units are linked together left, right, up, and down in a sheet form), or a one-dimensional network (e.g., the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic.

The term “cell migration modulator” refers to any agent that can modulate the migration route and/or rate of a cell or be used in or facilitate modulation of a migration route and/or rate of a cell. In some embodiments, the cell migration modulator is a cell attractant.

As used in this context herein a “cell attractant” refers to any agent that can cause cells to migrate towards and/or increase the rate of migration towards the cell attractant. In some embodiments, the cell migration modulator is a cell repellant. As used in this context herein a “cell repellant” refers to any agent that can cause cells to migrate away from and/or increase the rate of migration away from the cell attractant. In some embodiments, the cell attractant is cancer stem cell attractant (i.e., attracts cancer stem cells); a circulating cancer cell attractant (i.e., attracts circulating cancer cells); a migrating cancer cell attractant (i.e., attracts migrating cancer cells); a disseminating cancer cell attractant (i.e., attracts disseminating cancer cells); a glioma cell attractant (i.e., attracts glioma cells); a tumor microenvironment cell attractant (i.e., attracts cells in the tumor microenvironment cells); an immune cell attractant (i.e., attracts immune cells); a cancer cell attractant (i.e., attracts cancer cells); a cancer-associated fibroblast attractant (i.e., attracts cancer-associated fibroblast cells); and/or a tumor initiating cell attractant (i.e., tumor initiating cell attractant). In some embodiments a cell repellant repels cells that are not intended or desired to be captured by the injectable hydrogel. Such cells can be normal healthy and/or non-tumor and/or non-cancer cells. In some embodiments, such cells are normal brain cells, neurons and/or supporting cells (e.g., Schwann cells, astrocytes, astroglial, cells, microglial cells, dendritic cells, and/or the like). In some embodiments, combinations of cell attractants and repellants can be included in the injectable hydrogel so as to attract cells desired to be captured in the hydrogel and repel those that are not desired to be captured in the hydrogel.

As used herein, “hydrogel” refers to a three-dimensional network of hydrophilic polymers capable of swelling in an aqueous solution and are capable of holding water while maintaining the structure due to the cross-linking of the individual polymer chains that form a hydrogel matrix. Hydrogels can be gelatinous colloid or aggregate of polymeric molecules in a finely dispersed semi-solid state, where the polymeric molecules are in the external or dispersion phase and water (or an aqueous solution) forms the internal or dispersed phase. Hydrogels can be physical, chemical, or biochemical. Physical gels can undergo a transition from liquid to a gel in response to a change in environmental conditions such as temperature, ionic concentration, pH, or other conditions such as mixing of two components. Chemical gels use covalent bonding that introduces mechanical integrity and degradation resistance compared to other weak materials. In biochemical hydrogels, biological agents, such as enzymes, participate in the gelation process. Depending on the ionic charges on the bound groups, hydrogels can be cationic, anionic, or neutral. Cross-linking present in a hydrogel can be physical crosslinking or chemical crosslinking. Hydrogels can be amorphous, semicrystalline, crystalline, or hydrocolloid aggregates. Hydrogels are capable of undergoing significant volume phase transition or gel-sol phase transition in response to various stimuli, including physical and chemical stimuli. The transition can be irreversible or reversible. Where reversible, the reverse transition can occur via removal of the stimulus causing the transition and/or application of another stimulus to the hydrogel. Based on the methods of synthesis, hydrogels can be homopolymer hydrogels, semi-interpenetrating network (Semi-IPN), or interpenetrating network (IPN). Homopolymer hydrogels only contain one type of monomer in their structure. Copolymeric hydrogels are composed of two or more types of monomers (e.g., HEMA and TEGDMA), in which at least one is hydrophilic (e.g., poly(e-caprolactone)-HEMA macromonomer hydrogels). A semi-IPN forms when a linear polymer penetrates into another cross-linked network without any other chemical bonds between them. Semi-IPNs can more effectively preserve rapid kinetic response rates to pH or temperature due to the absence of a restricting interpenetrating elastic network while still providing the benefits like modified pore size, slow drug release, etc. Hydrogels can be composed of self-assembling peptides. Self-assembling peptide systems that are synthetic amino acid-based molecules which undergo a sol-gel transition when brought to neutral pH and ionic concentration. These systems do not use cross-linking agents; hence, they can safely encapsulate cells and/or drugs without exposing them to toxic agents (see e.g., D. Macaya, M. Spector, Injectable Hydrogel Materials for Spinal Cord Regeneration: A Review, Biomed. Mater. 2012; 7(1): 012001, DOI: 10.1088/1748-6041/7/1/012001).

As used herein, “attached” refers to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-πinteractions, cation-π interactions, anion-π interactions, polar π-interactions, and hydrophobic effects.

As used herein, “cell type” refers to the more permanent aspects (e.g. a hepatocyte typically can't on its own turn into a neuron) of a cell's identity. Cell type can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160.

As used herein, the term “effective proximity” or “operable proximity” used interchangeably herein refer to the distance, region, or area surrounding a reference point or object in which a desired effect or activity occurs. The effective proximity can be determined by measuring the desired effect or activity in a representative number of species in the area surrounding the reference point or object. By way of non-limiting examples, an agent can be delivered to a specific point in a tissue of a subject and can be diffused through the surrounding tissue and cause effects in cells at a distance from the initial point of delivery. Cells that are affected by the agent can be determined and thus the region of effective proximity can be determined. Cells within that region are said to be within effective proximity to the initial delivery point. Similarly, if a cell is engineered to produce a product and secretes it into the surrounding environment, cells in the surrounding environment that are affected by the secreted product are said to be within effective proximity to the producing cell (or reference point). In other non-limiting embodiments, a stimulus source (e.g. an electrical probe or ultrasound probe or light source, and/or the like) is said to be placed in effective proximity when the are placed within a distance of a location and/or object to which the stimulus is intended to affect or reach that causes the stimulus to reach, be delivered to, and/or achieve an effect at that location and/or object. In some embodiments, effective proximity can range from 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000 Angstroms, pm, nm, microns or mm away from the reference point.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

As used herein, “immunomodulator,” refers to an agent, such as a therapeutic agent, which is capable of modulating or regulating one or more immune function or response.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein, the term “radiation sensitizer” refers to agents that can selectively enhance the cell killing from irradiation in a desired cell population, such as tumor cells, while exhibiting no single agent toxicity on tumor or normal cells.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer, a reoccurring cancer, proliferative disease, and/or the like (including, but not limited to, solid tumor cancers, gliomas, glioblastoma. GBM, and other brain cancers). The effect can be therapeutic in terms of a partial or complete cure of a disease, condition, symptom or adverse effect attributed to the disease, disorder, or condition. The term “treatment” as used herein covers any treatment of a cancer, a reoccurring cancer, proliferative disease, and/or the like (including, but not limited to, solid tumor cancers, gliomas, glioblastoma. GBM, and other brain cancers). in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “proliferative disease” generally refers to any disease or disorder characterized by neoplastic cell growth and proliferation, whether benign, pre-malignant, or malignant. The term proliferative disease generally includes all transformed cells and tissues and all cancerous cells and tissues. Proliferative diseases or disorders include, but are not limited to abnormal cell growth, benign tumors, premalignant or precancerous lesions, malignant tumors, and cancer.

As used herein, “gene or genetic modifying agent” refers to any molecule, protein, or system capable of modifying the polynucleotide sequence of a nucleic acid, particularly one within a cytoplasm and/or nucleus of a cell. Such agents are generally known in the art and include, without limitation mega nucleases, zinc finger nucleases, ribozymes, CRISPR-Cas systems, transposons, CRISPR-Associated transposon systems, and the like.

As used herein “tumour” or “tumour tissue” refer to an abnormal mass of tissue resulting from excessive cell division. A tumour or tumour tissue comprises “tumour cells” which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumour or tumour tissue.

As used herein, the term “tumor microenvironment” (TME) refers to is the cellular environment in which the tumor exists, including surrounding blood vessels, immune cells, cancer associated fibroblasts (CAFs), bone marrow-derived inflammatory cells, lymphocytes, signaling molecules and the extracellular matrix (ECM).

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

### Overview

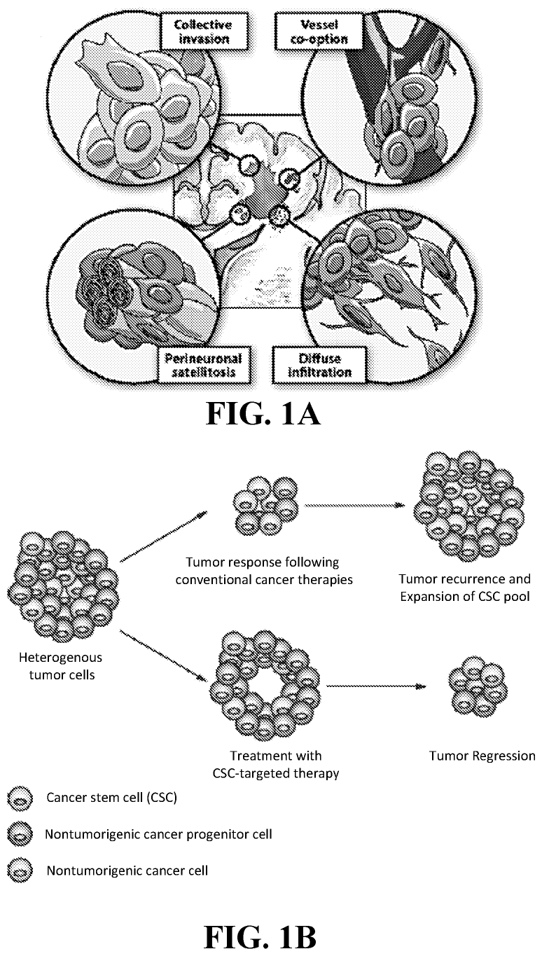

Glioblastoma multiforme (GBM) is the most aggressive type of primary brain tumor in adults. Its infiltrative capacity allows GBM to utilize a variety of strategies to diffuse to surrounding brain tissues. See e.g., FIG. 1B. Current treatments employ “search and destroy” strategies such as surgery, chemotherapy, and radiotherapy to eradicate GBM. See e.g., FIG. 1A. However, the glioma cancer stem cell (GSC) population is resistant to therapy remain, which leads to tumor recurrences and the poor prognosis of the disease. As such, there exists a need for improved treatments for GBM and other cancers, such as those where cancer stem cells play roles in continuation of the disease.

More recent developments in therapy options for GBM include an “attract to kill” strategy in which GBM cell migration is guided into scaffolds for entrapment (see e.g., Autier, L. et al. Acta biomaterialia. 84: 268-279, doi:10.1016/j.actbio.2018.11.027 (2019); van der Sanden, B. et al. Future oncology (London, England). 9:817-824, doi:10.2217/fon.13.30 (2013); Jain, A. et al. Nature materials 13, 308-316, doi:10.1038/nmat3878 (2014)). A failure of these approaches s that these scaffold materials do not target GSCs, and thus fail to prevent/treat recurrences.

With that said, embodiments disclosed herein can provide injectable hydrogels that, in some embodiments, are capable of attracting cells to them in situ. The injectable hydrogels can then be inserted into a cavity, such as a surgical cavity produced after tumor removal. This can be followed by delivering an energy to the injected hydrogel which can result in damage or killing of the cells present in the injected hydrogel. In some embodiments, the cells attracted to, captured by and/or present in the injected hydrogel are cancer cells, such as cancer stem cells. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Injectable Hydrogels

Described in several example embodiments herein are injectable hydrogels. The injectable hydrogels can be configured to capture and retain cells that are induced to migrate to the injectable hydrogel when in use within a subject. In some embodiments, the injectable hydrogels can be configured for use with a first external stimulus that is effective to induce migration of cells, optionally in a cell selective manner, to the hydrogel when in use within a subject. In some embodiments, the injectable hydrogels contain one or more agents that are effective to modulate the migration of one or more cells, such as to attract cells, optionally in a cell type selective manner to the hydrogel and/or repel cells, optionally in a cell type selective manner away from the hydrogel. Optionally, the hydrogels can be responsive to an external stimulus that is capable of causing release of one or more agents contained within the injectable hydrogel, such as a cell migration modulator and/or other agent contained within the injectable hydrogel. The hydrogel can be configured for use with a second external stimulus that is effective to modify, inhibit, stimulate, and/or kill one or more cells captured and/or retained within the cell. Effectively the injectable hydrogels can localize a desired cell population within a subject and allow for manipulation and/or killing of the localized cells. This can be particularly advantageous where modification, manipulation, and/or kill of those cells without localization in this way is extremely challenging or not possible.

### Injectable Hydrogel Compositions

In some embodiments, the injectable hydrogels contain a hydrogel matrix comprising one or more polymers, a wt % of water that is any non-zero wt % that ranges from about 0 up to, but not including, 75 wt %; optionally, one or more agents, wherein one of the one or more agents is a optionally cell migration modulator, wherein the storage modulus of the injectable hydrogel ranges from about 3 to about 100 kPa, about 3 kPa to about 50 kPa, or about 3 kPa to about 25 kPa. The one or more polymers can be crosslinked so as to form the hydrogel matrix. The crosslinking can be formed by any suitable method, chemical, photo, or physical. In some embodiments, the crosslinking is formed by a thiol-Michael addition or a thiol-Michael addition click reaction.

In some embodiments, the wt % of water content in the hydrogel is any non-zero wt % ranging from about 0 to about 50 wt %, 0 to about 25 wt %, about 25 wt % to about 50 wt %, about 25 wt % up to, but not including 75 wt %, or about 50 wt % up to, but not including, 75 wt %. In some embodiments, the wt % of water content in the hydrogel is any non-zero number or integer thereof that is about 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5 or any other number up to, but not including, 75 wt. % or range of values therein. In some embodiments, the wt % of water content in the hydrogel is any non-zero number or integer thereof that is about 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or/to 50 wt. %. In some embodiments, the wt % of water content in the hydrogel is any non-zero number or integer thereof that is about 0, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or/to 25 wt. % In some embodiments, the wt % of water content in the hydrogel is about 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or/to 50 wt. %. In some embodiments, the wt % of water content in the hydrogel is about 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5 or any other number up to, but not including, 75 wt. % or range of values therein. In some embodiments, the wt % of water content in the hydrogel is about 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5 or any other number up to, but not including, 75 wt. % or range of values therein.

The storage modulus (kPa) of the injectable hydrogel can range from about 3 kPa to about 100 kPa. In some embodiments, the storage modulus of the injectable hydrogel is about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, or about 100 kPa. In some embodiments, the storage modulus (kPa) of the injectable hydrogel ranges from about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, or 99.5, to about 100 kPa. In some embodiments, the injectable hydrogel has a storage modulus effective for implantation into brain tissue. The storage modulus (G′) of a hydrogel can be measured by any suitable method generally known in the art and as demonstrated in the Working Examples herein. Such methods and calculations will be readily appreciated by those of ordinary skill in the art.

In some embodiments, the swelling ratio ranges from about 0% to about 200% or more. In some embodiments, the swelling ratio is 10, 20, 30, 40, 50, 60, 70, 80, 90, 100, 110, 120, 130, 140, 150, 160, 170, 180, 190, to/or 200% or more. In some embodiments the hydrogels are optically transparent. The swelling ratio can be determined by methods known in the art.

The hydrogel matrix can be porous. The porosity can be formed by crosslinking or other connections made between the polymers of the hydrogel matrix. The pores can be any regular or irregular shape. The average longest dimension of the pores can range from about any-non number of about 0.001, 0.01, or about 0.1 nm or um to about 1,000 nm or um or more. In some embodiments the average pore size (also referred to in the Working Examples herein as “mesh size”) is about 3, 4, 5, 6, 7, 8, 9, 10, 11, 12, 13, 14 to/or about 15 nm. In some embodiments, the average pore size is about 3, 4, 5, 6, 7, 8, 9, 10, 11, 12, 13, to/or about 14 nm. In some embodiments, the average pore size is about 3, 4, 5, 6, 7, 8, 9, 10, 11, 12, to/or about 13 nm. In some embodiments, the average pore size is about 3, 4, 5, 6, 7, 8, 9, 10, 11, to/or about 12 nm. In some embodiments, the average pore size is about 3, 4, 5, 6, 7, 8, 9, 10, to/or about 11 nm. In some embodiments, the average pore size is about 3, 4, 5, 6, 7, 8, 9, to/or about 10 nm. In some embodiments, the average pore size is about 3, 4, 5, 6, 7, 8, to/or 9 nm. In some embodiments, the average pore size is about 3, 4, 5, 6, 7, to/or about 8 nm. In some embodiments, the average pore size is about 3, 4, 5, 6, to/or about 7 nm. In some embodiments, the average pore size is about 3, 4, 5, to/or about 6 nm. In some embodiments, the average pore size is about 3, 4, to/or about 5 nm. In some embodiments, the average pore size is about 3 to/or about 4 nm. The pore diameters and average pore diameters/sizes/dimensions can be determined by methods known in the art.

In some embodiments, the injectable hydrogel is cationic, nonionic, or anionic.

### Polymers

The hydrogel matrix is composed of one or more polymers that can be crosslinked, such as via a Thiol-Michael addition reaction or otherwise joined to form the matrix structure. In some embodiments, the one or more polymers (or monomers thereof) or at least one of the one or more polymers has one or more hydrophilic groups. In some embodiments, each of the one or more hydrophilic groups are individually selected from the group of: —NH₂, —COOH, —OH, —CONH₂, —CONH—, and —SO₃H. Each of the one or more polymers can be individually selected from a natural polymer and a synthetic polymer. In some embodiments, all the polymers in the hydrogel are natural polymers. In some embodiments, all the polymers in the hydrogel are synthetic polymers. In some embodiments, where two or more polymers are present, at least one polymer is a natural polymer and at least one polymer is a synthetic polymer.

In some embodiments, the one or more polymers are chemically crosslinked, physically crosslinked, or both. In some embodiments, some or all of the crosslinking are formed via a Thiol-Michael addition reaction, such as a Thiol-Michael addition click reaction.

In some embodiments, the one or more polymers are each individually selected from polyethylene glycol (PEG), chitosan, Poly(-hydroxyethyl methacrylate) (PHEMA), 2-Hydroxyethyl methacrylate (HEMA), Hydroxyethoxyethyl metha-crylate (HEEMA), Hydroxydiethoxyethylmethacrylate (HDEEMA), Methoxyethyl methacrylate (MEMA), Methoxyethoxyethyl methacrylate (MEEMA), Methoxy-diethoxyethyl methacrylate (MDEEMA), Ethylene glycol dimethacrylate (EGDMA), N-vinyl-2-pyrrolidone (NVP), N-isopropyl AAm (NIPAAm), Vinyl acetate (VAc), Acrylic acid (AA), N-(2-hydroxypropyl) methacrylamide (HPMA), Ethylene glycol (EG), PEG acrylate (PEGA), PEG methacrylate (PEGMA), PEG diacrylate (PEGDA), PEG dimethacrylate (PEGDMA), Methacrylic acid (MAA), PEG-PEGMA, Carboxymethyl cellulose (CMC), Polyvinylpyrrolidone (PVP), an Acrylamide/acrylic acid copolymer, linear cationic polyallylammonium chloride, Poly(N-isopropyl acrylamide) (PNIPAM), self-assembling peptides, acrylate-modified PEG and acrylate-modified hyaluronic acid, heparin, amine end-functionalized 4-arm star-PEG, or any combination thereof.

In some embodiments, at least one of the one or more polymers is PEGDA. In some embodiments, one or more of the polymers is PEGDA. In some embodiments, the hydrogel is only composed of PEGDA polymers.

The molecular weight of the polymers can be any suitable molecular weight. In some embodiments, the polymers present in the hydrogel each has an average molecular weight that is independently selected from about 100 to about 1000 Da, about 100 to about 900 Da, about 100 to about 800 Da, about 100 Da to about 700 Da, about 200 Da to 600 Da, about 300 Da to about 600 Da, about 400 Da to about 600 Da, about 500 Da to about 600 Da, about 525 Da to about 600 Da, about 550 Da to about 600 Da, 575 Da to about 600 Da. In some embodiments, the average MW of the polymers is about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or about 1000 Da. In some embodiments, the average molecular weight of the one or more polymers is about 500-600 Da. In some embodiments, the average molecular weight of the one or more polymers is about 575 Da. In some embodiments where two or more different polymers are included in the hydrogel matrix, at least two of the polymers have the same average molecular weight. In some embodiments where two or more different polymers are included in the hydrogel matrix, at least two of the polymers have a different average molecular weight. In some embodiments, where a single polymer is included, such as PEGDA, the polymer can be included at two or more different molecular weights (e.g., PEGDA with an average molecular weight of about 575 Da and PEGDA with an average molecular weight that is not 575 Da, such as 200 Da, 1000 Da or more). In some embodiments, where there is only a single polymer, the single polymer is included at a homogenous molecular weight. For example, in some embodiments the hydrogel matrix only includes PEGDA with an average molecular weight of about 575 Da.

### Agents

The injectable hydrogels can be loaded with and thus include one or more agents. The one or more agents can be released (either continuously or controlled), such as into the environment surrounding the hydrogel once injected/implanted. Release of the agent(s) from the hydrogel is described elsewhere herein.

The one or more of the one or more agents can each individually be selected from the group of: DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, agent sensitizers, imaging agents, chemotherapeutic agents, chemokines, cytokines, anti-migratory compounds capable of inhibiting chemokine receptors to decrease cell invasion, and any combination thereof.

An agent can be a hormone. Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol). Exemplary hormones include, without limitation, endothelin, exendin, follicle stimulating hormone, growth hormone releasing hormone, growth hormone releasing peptides, ipamorelin, glucagon, glucagon-like peptides, insulin, chorionic gonadotropin, inhibin-Beta C Chain, inhibin alpha, inhibin alpha chain, luteinizing hormone, luteinizing hormone releasing hormone, peptide hormones (e.g., adrenocorticotropic hormone, alarelin, antide, atosiban, buserelin, cetrorelix, desmopressin, deslorelin, elcatonin, ganirelx, ghrelin, goserelin, hexarelin, gistrelin, lanreotide, leuprolide, lypressin, melanotan-I and -II, nafarelin, octreotide, pramlintide, secretin, sincalide, somatostatin, terlipressin, thymopentin, triptorelin, vasopressin, neuropeptide Y, cholecystokinin), procalcitonin, prolactin, oxytocin, parathyroid hormone, estrogen, testosterone, stanniocalcin-1 and -2, thymosin, thyrostimulin, thyroid stimulating hormone, agouti-related protein, calcitonin, corticotrophin releasing hormone binding protein, prouroguanylin, oxyntomodulin, thyrotripin releasing hormone, and any combination thereof.

An agent can be an immune modulator. Suitable immunomodulators include, but are not limited to, prednisone, azathioprine, 6-MP, cyclosporine, tacrolimus, methotrexate, interleukins (e.g., IL-2, IL-7, IL-6, and IL-12), cytokines, chemokines, cytosine phosphate-guanosine, oligodeoxynucleotides, glucans, antibodies, and aptamers. In some embodiments, one or more of the one or more agents is a chemokine. Exemplary chemokines include, without limitation, e.g., CCL3, CCL26, CXCL13, CXCL14, CCL6, CCL27, CXCL16, CXCL17, CXCL6, CXCL5, eotaxin, CCL2, CX3CL1, CXCL1, 2, 3, CCL14, CCL1, CXCL8, CXCL11, CC3L1, XCL1, CCL2, 7, 8, 12, 13, CCL22, CCL28, CXCL9, CCL3, 4, 9, 15, CXCL7, CCL4, CXCL4, CXCL12, CCL17, CCL25, CCL16, FAM19A5, CXCL15, and any combination thereof. In some embodiments, one or more of the one or more agents is a cytokine. Exemplary cytokines include, without limitation, interferons (e.g. IFN-a, IFN-β, IFN-ε, IFN-K, IFN-ω, and IFN-γ), granulocyte colony-stimulating factor, imiquimod, 4-1BB, adiponectin, AITR, AIF1, B-cell activating factor, beta defensin, betacellulin, BMP, BST1, B type Natriuretic peptide, cardiotrophin, CTLA4, EBI3, Endoglin, epiregulin, FAS, Flt3 ligand, follistatin, hedgehog protein, interferons (e.g. interferon alpha, interferon gamma, interferon tau, interferon beta, interferon regulatory factor), interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-8, IL-19, IL-20, IL-21, IL-22, IL-23, IL-24, IL-27, IL-28A, IL-29, IL-31, IL-32, IL-33, IL-34, IL-35, IL-36, IL-37), otoraplin, resistin, leukemia inhibitory factor, serum amyloid A, TPO, trefoil factor, thymic stromal lymphopoietin, tumor necrosis factor, uteroglobin, visfatin, wingless-type MMTV nitration site family, AIMP1, CLCF1, CYTL1, EMAP II, TAFA2, Vaspin, and any combination thereof.

An agent can be an antipyretic. Suitable antipyretics include, without limitation, non-steroidal anti-inflammatories (e.g., ibuprofen, naproxen, ketoprofen, nimesulide, diclofenac, diflunisal, etodolac, indomethacin, ketorolac, nabumetone, oxaprozin, piroxicam, salsalate, sulindac, tolmetin, celecoxib, valdecoxib, firocoxib, and rofecoxib), aspirin and related salicylates (e.g., choline salicylate, magnesium salicylate, and sodium salicylate), paracetamol/acetaminophen, metamizole, phenazone, and quinine.

An agent can be an anxiolytic. Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotonergic antidepressants (e.g. selective serotonin reuptake inhibitors, serotonin-norepinephrine reuptake inhibitors, tricyclic antidepressants (e.g. imipramine, doxepin, amitriptyline, nortriptyline, and desipramine), tetracyclic antidepressants (e.g. mirtazapine) and monoamine oxidase inhibitors (e.g., phenelzine, isocarboxazid, and tranylcypromine), mebicar, fabomotizole, selank, bromantane, emoxypine, azapirones, barbiturates, hydroxyzine, pregabalin, validol, beta blockers (e.g., acebutolol, atenolol, betaxolol, bisoprolol, carteolol, carvedilol, Propofol, racetam-based drugs (e.g., aniracetam), alcohol, esmolol, labetalol, metoprolol, nadolol, nebivolol, penbutolol, pindolol, propranolol, sotalol, and timolol), and carbamates (e.g., meprobamate, tybamate, lorbamate).

An agent can be an antipsychotic. Suitable antipsychotics include, but are not limited to, benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, clomipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, bifeprunox, bitopertin, brexpiprazole, cannabidiol, cariprazine, pimavanserin, pomaglumetad methionil, vabicaserin, xanomeline, and zicronapine.

An agent can be an analgesic. Suitable analgesics include, but are not limited to, paracetamol/acetaminophen, non-steroidal anti-inflammants (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

An agent can be an antispasmodic. Suitable antispasmodics include, but are not limited to mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene.

An agent can be an anti-inflammatory agent. Suitable anti-inflammatories include, but are not limited to, prednisone, non-steroidal anti-inflammants (e.g., ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

An agent can be an antihistamine. Suitable antihistamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and β2-adrenergic agonists.

An agent can be an anti-infective. As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals. Exemplary anti-infectives include without limitation amebicides (e.g., nitazoxanide, paromomycin, metronidazole, tinidazole, chloroquine, and iodoquinol), aminoglycosides (e.g., paromomycin, tobramycin, gentamicin, amikacin, kanamycin, and neomycin), anthelmintics (e.g., benzimidazoles (e.g., albendazole, mebendazole, thiabendazole, fenbendazole, triclabendazole, flubendazole) abamectin, ivermectin, diethylcarbamazine, pyrantel pamoate, levamisole, silicylanilides (e.g., niclosamide, oxyclozanide), nitazoxanide, praziquantel, octadepsipeptides (e.g., emodepside), monepantel, spiroindoles (e.g., derquantel), artemisinin, moxidectin, milbemycins (e.g., milbemycin oxime), antifungals (e.g. azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceftaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzathine and benzylpenicillin), phenoxymethylpenicillin, cloxacillin, flucloxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefixime, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, aztreonam, tigemonam, nocardicin A, taboxinine, and beta-lactam), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

An agent can be a chemotherapeutic. Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g. cisplatin, carboplatin, and oxaliplatin), gemcitabine, DNA topoisomerase I-targeting drugs (e.g. camptothecin derivatives (e.g. topotecan and irinotecan)), epidermal growth factor receptor blockade family agents (e.g. cetuximab, gefitinib), farnesyltransferase inhibitors (e.g., L-778-123), COX-2 inhibitors (e.g. rofecoxib, celecoxib, and etoricoxib), bFGF and VEGF targeting agents (e.g. bevazucimab and thalidomide), NBTXR3, Nimoral, trans sodium crocetinate, NVX-108, and combinations thereof. See also e.g., Kvols, L. K., J Nucl Med 2005; 46:1875-1905.

In some embodiments, an agent is a biologic agent. As used herein, “biologic agent” refers to any compound, composition, biopolymer, molecule and the like that is made by a living organism and include, without limitation, polynucleotides (e.g., DNA, RNA), peptides and polypeptides, and chemical compounds (e.g., hormones, chemokines, and cytokines). In some embodiments, the biologic agent can be an antibody or fragment thereof. As used herein, “antibody” refers to a protein or glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies.

In some embodiments, the agent is a cell or cell population. In some embodiments, the cell or cell population is an adoptive cell therapy (i.e., the cells were obtained from a subject and manipulated and/or engineered ex vivo to be delivered to a patient from which they were derived (e.g., autologous adoptive therapy) or a different recipient from which they were derived (e.g., allogenic adoptive therapy). In some embodiments, the cell or cell population is an engineered CAR cell (e.g., a CAR-T and/or CAR-NK cell). In some embodiments, the engineered CAR cell is active against a brain tumor. In some embodiments, the brain tumor is a glioblastoma. See e.g., Bagley et al., Neuro Oncol. 2018. 20(11):1429-1438, doi: 10.1093/neuonc/noy032; Patterson et al., Front Oncol. 2020 Aug. 12; 10:1582. doi: 10.3389/fonc.2020.01582. eCollection 2020; Choi et al., Clin Cancer Res. 2019 Apr. 1; 25(7):2042-2048. doi: 10.1158/1078-0432.CCR-18-1625. Epub 2018; Akhavan et al., Immunol Rev. 2019 July; 290(1):60-84. doi: 10.1111/imr.12773; Li et al. Front Immunol. 2020 Nov. 3; 11:594271. doi: 10.3389/fimmu.2020.594271. eCollection 2020; and Burger et al., Front Immunol. 2019 Nov. 14; 10:2683. doi: 10.3389/fimmu.2019.02683. eCollection 2019).

An agent can be a gene modifying agent. Exemplary gene modifying agents include, but are not limited to, RNA guided or programmable nuclease systems such as a CRISPR-Cas system, Meganucleases, Zinc Finger Nucleases, and/or the like. Such systems are generally known in the art.

As used herein, “deoxyribonucleic acid (DNA)” and “ribonucleic acid (RNA)” generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

In some embodiments, an agent is a chemical agent. As used herein, “chemical agent” refers to a chemical substance, molecule, or composition. Exemplary chemical agents are those that are suitable for use as a pharmaceutical agent in an animal as well as those that are not. In some embodiments, the chemical agent is a hazardous chemical agent. In some embodiments, the chemical agent is not hazardous. In some embodiments, the chemical agent can be a carcinogen. In some embodiments, the chemical agent is biocompatible. The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

In some embodiments, an agent can be a pharmaceutical agent. As used herein, “pharmaceutical agent” refers to any compound, molecule, or composition that is capable of preventing, treating, diagnosing, and/or prognosing a disease, condition, disorder, or any symptom thereof. Pharmaceutical agents can be of any type, including without limitation chemical agents and biologic agents.

In some embodiments, an agent is a growth factor. Exemplary growth factors include without limitation vascular endothelial growth factor (VEGF), bone morphogenetic protein(s) (BMP), a transforming growth factor (TGF) such as transforming growth factor beta, a platelet derived growth factor (PDGF), an epidermal growth factor (EGF), a nerve growth factor (NGF), an insulin-like growth factor (e.g., insulin-like growth factor I), scatter factor/hepatocyte growth factor (HGF), granulocyte/macrophage colony stimulating factor (GMCSF), a glial growth factor (GGF), and a fibroblast growth factor (FGF), GCSF, Erythropoietin, TPO, GDF, neurotrophins, MSF, SGF, GDF, activin, CTGF, Epigen, Galectin, KGF, leptin, MMIF, MIA (melanoma inhibitory activity), myostatin, noggin, NOV, omentin, Oncostatin-M, Osteopontin, OPG, periostin, placental growth factor, placental lactogen, prolactin, RNAK ligand, retinol binding protein (RBP), stem cell factor, amphiregulin, lymphocyte function associated Antigen-3, myeloid derived growth factor, osteoclast stimulating factor, progranulin, colony stimulating factor and combinations thereof.

In some embodiments, an agent is an inhibitor of cancer stem cells, including but not limited to GSCs, CSCs (circulating stem cells). Exemplary cancer stem cell inhibitors include, but are not limited to, salinomycin, antibodies targeted to the GSCs and/or CSCs (e.g., monoclonal antibodies targeting e.g., GSCs and/or CSC-specific surface epitopes, e.g., EpCAM, CD123, CD16, CD20, CD52, CD19, CD22, and/or ALDH), aptamers targeted to the GSCs and/or CSCs, CAR cells targeted to the GSCs; Wnt signaling pathway inhibitors (e.g., ipafricept, PRI-724, CWP232291, LGK974 (NCT02278133), ETC-159 (NCT02521844), ETC-1922159, and Vantictumab (OMP-18R5) (NCT01973309, NCT01957007, and NCT02005315)), Notch signaling pathway inhibitors (e.g., gamma secretion inhibitors (e.g., MK-0752 (NTC00100152), R04929097, NirogacestatPF-03084014, NCT01154452, BMS-906024 (NCT01292655), BMS-986115 (NCT01986218), CB-103 (NCT03422679), Crenigacestat LY3039478 (NCT02836600), AL101, BMS-906024, and LY900009 (NCT01158404)), Notch signaling pathway and/or receptor antibodies or ligands (e.g., Demcizumab (OMP-21M18), Brontictuzumab (OMP-52M51), MEDI0639), DLL4 inhibitors (e.g., IgG2 mAb targeting DDL4, Enoticumab, and DLL4 inhibitors as in clinical trial NCT02259582)), Hh signaling pathway inhibitors (e.g., vismodegib (GDC-0449), sonidegib (LDE225), and glasdegib (PF-04449913)), TGF-beta signaling pathway inhibitors ((e.g., Galunisertib (LY2157299), LY3200882, AVID200, Trabedersen (AP 12009), Fresolimumab (GC1008), Vactosertib (TEW-7197), and NIS793); JAK-STAT pathway signaling inhibitors (e.g., Ruxolitinib, AZD4205, SAR302503, and SB1518); PI3K inhibitors (e.g., Alpelisib, Buparlisib (BKM120), BYL719, SF1126, and SAR245409); EGFR inhibitors (e.g., Bevacizumab and Matuzumab (EMD 72000)), metabolism inhibitors (e.g., Venetoclax (ABT-199), CB-1158, Telaglenastat, IM156, TVB-2640, Rifampicin, 131I-TLX-101, and Pegzilarginase); Niche inhibitors (e.g., Plerixafor (Mozobil), BL-8040, BKT140, BMS-936564, BMS-936564, LY2510924, MSX-122, USL311, AMD3100, Reparixin, and Defactinib (VS-6063); NF-kappaB signaling pathway inhibitors, mitochondrial glycolysis pathway inhibitors (e.g., Venetoclax, VIA-2291, GSK2190915), hedgehog inhibitors (e.g., Vismodegib (GDC-0449), Sonidegib (LDE225), Glasdegib, BMS-833923 (XL139), Taladegib (LY2940680), LEQ-506, G-024856, Patidegib (IPI-926), and any combination thereof.

An agent can be a CSC or GSC microenvironment inhibitor. Exemplary CSC or GSC microenvironment inhibitor include, but are not limited to, CXCR4 targeting agents (e.g., plerixafor (AMD3100), LY2510924 (alone or in combination with one or more other agents such as those specified in clinical trials (NCT03746080, NCT01977677, NCT01288573, NCT00103662, NCT01220375, and NCT00903968).

In some embodiments, an agent is an anti-migratory compound capable of inhibiting chemokine receptors to decrease cell invasion and serves as a chemokine antagonist, which include but are not limited to Met-CCL5, CCX721, BL5923, CCX9588, AMD3100, POL5551, AMD3465, and LY2510924. Some anti-migratory compounds are also capable of targeting actin polymerization in cells by destabilizing actin filaments to promote actin aggregation, which include but are not limited to cucurbitacin E, jasplakinolide, and chondramide, as well as agents that destabilize actin cytoskeletons like geodiamolides, cytochalasins, and latrunculins. Natural compounds, such as Neobractatin, can also be utilized to affect the pAKT/EMT pathway in cancer metastasis and inhibit cancer cell invasion. In some embodiments, the antimigratory compound(s) is/are AMD33465, POL5551, or both. See also, Robinson et al. Cancer Res. 2003. 63(23); 8360-8365; Poeta et al., Font. Immunol. 2019. 10:379; Gandalovicova et al., Trends Cancer. 2017 June; 3(6):391-406. doi: 10.1016/j.trecan.2017.04.008, particularly at Table 1, and Zhang at al., Cell Death &. Disease. 10 Article No. 554 (2019).

In some embodiments, at least one of the one or more agents is a cell migration modulator. Cell migration modulators can be chemical or biological agents and thus modulate chemotaxis of cells. Cell migration modulators can be an energy, such as a magnetic field, light, or electrical energy and thus modulate the photo-, magnetic-, or electrotaxis of a cell. The cell migration modulator can act to attract a cell, such as a GBM and/or GSC and/or CSC. In some embodiments, the migration modulator selectively attracts GBM and/or GSC, and/or CSC cells. In some embodiments, the cell migration modulation is a chemokine, cytokine, a GBM and/or GSC cell receptor ligand. Exemplary ligands and antibodies are presented above, with respect to GSC/CSC inhibitors. Such receptor ligands and antibodies can be used not only to inhibit or kill GSC/CSCs but used to target the cells to the hydrogel and/or capture them within the hydrogel. In some embodiments, the cell migration modulator is a chemokine or cytokine. Exemplary, non-limiting, chemokine and cytokines are presented above as exemplary agents and can act as cell migration modulators. In some embodiments, the cell migration modulator is CX3CL1, CXCL12, CXCL7, CXCL8, fMLE, Annexin 1, and combinations thereof.

### Agent Amounts

Each of the one or more agents included in the hydrogel can be included at any appropriate or suitable amount, including an effective amount or least effective amount. Such an amount can be determined empirically, such as by kinetic analysis of release profile, loading profile, etc. As used herein, “effective amount” refers to the amount of an agent included in the hydrogel that achieve one or more therapeutic effects or desired effect. As used herein, “least effective” amount refers to the lowest amount of an agent included in the hydrogel that achieves the one or more therapeutic or other desired effects. As used herein, “therapeutically effective amount” refers to the amount of the agent included in the hydrogel that achieves one or more therapeutic effects. Techniques to determine such amounts are generally known in the art and can be utilized by one of ordinary skill in the art in view of the description herein. Unless specified otherwise herein amounts are measured after loading but before use and/or release of any agents.

In some embodiments, the amount, effective amount, least effective amount, and/or therapeutically effective amount of an agent included in the hydrogel can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the amount, effective amount, least effective amount, and/or therapeutically effective amount of an agent included in the hydrogel can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In some embodiments, the amount, effective amount, least effective amount, and/or therapeutically effective amount of an agent included in the hydrogel can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the amount, effective amount, least effective amount, and/or therapeutically effective amount of an agent included in the hydrogel 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as an agent), the effective amount of cells can range from about 2 cells to 1×10¹/mL or cc of hydrogel, 1×10²⁰/mL or cc of hydrogel or more, such as about 1×10¹/mL, 1×10²/mL or cc of hydrogel, 1×10³/mL or cc of hydrogel, 1×10⁴/mL or cc of hydrogel, 1×10⁵/mL or cc of hydrogel, 1×10⁶/mL or cc of hydrogel, 1×10⁷/mL or cc of hydrogel, 1×10⁸/mL, 1×10⁹/mL or cc of hydrogel, 1×10¹⁰/mL or cc of hydrogel, 1×10¹¹/mL or cc of hydrogel, 1×10¹²/mL or cc of hydrogel, 1×10¹³/mL or cc of hydrogel, 1×10¹⁴/mL or cc of hydrogel, 1×10¹⁵/mL or cc of hydrogel, 1×10¹⁶/mL or cc of hydrogel, 1×10¹⁷/mL or cc of hydrogel, 1×10¹⁸/mL or cc of hydrogel, 1×10¹⁹/mL or cc of hydrogel, to/or about 1×10²⁰/mL or cc of hydrogel.

In some embodiments, the amount or effective amount, particularly where an infective particle is being delivered (e.g., a virus particle that is an agent and/or having an agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

Methods of loading hydrogels with agents are generally known in the art. In some embodiments, the one or more agents are mixed with a hydrogel solution as the hydrogel is being fabricated. The agents are then captured inside the hydrogel matrix when it solidifies.

### Eluting and Responsive Hydrogels

In some embodiments, the injectable hydrogel is capable of releasing or eluting one or more agents contained in the hydrogel into the environment outside of the hydrogel, such as into a cavity into which the hydrogel is placed. In some embodiments, the elution can be automatic and continuous (i.e., not necessarily in direct response to a stimulus applied to the injectable hydrogel). In some embodiments, the hydrogel is a responsive hydrogel (i.e., is responsive to one or more stimuli applied to the hydrogel, where the response can be a change in one or more characteristics of the hydrogel such that one or more agents are eluted/released from one or more regions of the hydrogel into the environment surrounding the hydrogel. Responses include swelling and shrinking, eluting one or more agents from the hydrogel, or a combination thereof.

In some embodiments, the stimulus is an abiotic environmental condition (e.g., pH, salinity, osmolarity, temperature (heat/cold), redox potential/differences, and/or the like, and/or any combination thereof) a chemical (e.g., a chemical agent(s)), a biologic agent (e.g., a biologic molecule such as a protein (e.g., an enzyme) and/or the like, and/or any combination thereof), an energy (thermal energy, electric energy, acoustic energy, light energy, magnetic field, radiation energy, and/or the like, and/or any combination thereof), a physical stimulus (e.g., a mechanical stress, pressure, strain, and/or the like, and/or any combination thereof), or any combination thereof.

In some embodiments, the injectable hydrogel is an agent eluting hydrogel and is capable of releasing one or more agents into the environment surrounding the hydrogel.

In some embodiments, the stimulus is capable of triggering agent elution from the hydrogel, agent activation, agent deactivation, or any combination thereof.

In some embodiments, the hydrogel is configured as a controlled release hydrogel (i.e., a hydrogel in which elution or release of an agent therein is controlled, optionally by an external stimulus and/or via the hydrogel matrix composition, over a period of time. In some embodiments, the rate of release is constant. In some embodiments the rate of release is variable. With respect to the hydrogel matrix composition, hydrogels with high porosity can be obtained by controlling two factors: the degree of cross-linking in the matrix and the affinity of hydrogel to the aqueous environment in which swelling occurs. Due to the porous structures, hydrogels are highly permeable to different kinds of drugs and thus drugs can be loaded and, in proper conditions, released (see e.g., M. Bahram, et al., J. Iran. Chem. Soc. 2015; 12(10): 1781-1787, DOI: 10.1007/s13738-015-0653-5. The possibility of releasing agents for long periods of time (i.e., sustained release) is an advantage obtained from hydrogels in delivery applications, which can result in supplying a high concentration of an active agent to a specific location over a long period of time.

Both physical (e.g., electrostatic interactions) and chemical (e.g., covalent bonding) strategies can be employed to enhance the binding between a loaded drug and the hydrogel matrix to extend the duration and/or slow the rate of agent release. Hydrogels can store and protect various agents contained therein from hostile environments and release them at a desired kinetics of the release. In some embodiments, agent release can be optionally activated on demand by an external stimulus including, but not limited to, local changes in pH, temperature, the presence of specific enzymes, or by remote physical stimuli. In some embodiments, the hydrogel is not responsive to one or more stimuli. In some embodiments, the hydrogel is selectively responsive to one or more stimuli but not others. For example, the hydrogel can be responsive to temperature, such as a physiologic temperature such that the one or more agents are released when present in a body cavity in a subject but is not responsive to one or more energies such as light, acoustic, or electrical used to ablate cells that are captured within an injected hydrogel. In some embodiments, the hydrogel is electrically conductive but not necessarily responsive to an electrical energy. In some embodiments, the hydrogel is electrically conductive and responsive to an electrical energy. In some embodiments, the hydrogel is electrically conductive but not necessarily responsive to an electrical energy and not responsive to an acoustic energy, such as ultrasound. In some embodiments, the hydrogel is electrically conductive and responsive to an electrical energy and not responsive to an acoustic energy, such as ultrasound.

The hydrogel can be configured such that it releases one or more agents continuously (e.g., without the aid of a stimulus) or responsively (i.e., in response to a stimulus), or both (e.g., release is continuous but can be further controlled by a stimulus, such as an electrical energy, which can act to increase the rate of release). In some embodiments, the release rate, amount, etc. is the same for two or more agents present in the hydrogel. In some embodiments, the release rate, amount, etc. is different for two or more agents present in the hydrogel. It will be appreciated by those of ordinary skill in the art that the composition of the agent can affect the release kinetics of the agent. Release kinetics can be measured by standard techniques known in the art and demonstrated in the Working examples herein. In some embodiments, the hydrogel can release one or more agents into the environment surrounding the hydrogel (such as tissue of a subject around the cavity in which it is injected/implanted) without a stimulus or in response to a stimulus for a period of time ranging from about 0.5 hours to 12 h, 18 hr, 24 hr, 36 hr, 48 hr, or 72 h or more. In some embodiments, the hydrogel can release one or more agents into the environment surrounding the hydrogel (such as tissue of a subject around the cavity in which it is injected/implanted) without a stimulus or in response to a stimulus for a period of time ranging from 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days, months, or years. In some embodiments, a stimulus is continually applied (e.g., such as body temperature or another constant abiotic environmental stimulus) so long as the hydrogel is implanted. In some embodiments, release is only during and/or for a non-perpetual time period following application of a stimulus to the hydrogel. The non-perpetual time period following application of a stimulus can range about 0.5 hours to 12 h, 18 hr, 24 hr, 36 hr, 48 hr, or 72 h or more. The non-perpetual time period following application of a stimulus can range about 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days, months, or years.

In some embodiments, the injectable hydrogel is responsive to an energy stimulus. The energy stimulus can be a light energy, electrical energy, thermal energy (see also temperature responsive hydrogels), radiation energy, acoustic energy, magnetic field, and/or the like). In some embodiments, a light energy is in the ultraviolet (UV) wavelength range. In some embodiments, a light energy is in the near infrared wavelength range. In some embodiments, a light energy is in the far infrared wavelength range. In some embodiments, a light energy is in the visible light wavelength range. In some embodiments, the energy is or includes an acoustic energy. In some embodiments the acoustic energy is ultrasound. In some embodiments, the ultrasound is a low frequency ultrasound. In some embodiments, the ultrasound is within about 0.1-50, 0.5-50, 1-25, 1-20, 1-18, 1-15, 1-10, 1-5, or about 1-3 megahertz. Although ultrasound can be used in a release context, in some embodiments this may not be desirable. For example, if ultrasound or other ablative therapy is used for cell ablation of captured cells, it may not be desirable for the cell to be responsive and release contents of the hydrogel. Therefore, in some embodiments, the hydrogel is not responsive to an energy, such as ultrasound or other ablative energy.

In some embodiments, the injectable hydrogel is pH responsive. In other words, the hydrogel is responsive to a pH stimulus. In some embodiments, the pH stimulus is an acidic pH (0 up to 7), a neutral pH (i.e., pH of 7), and/or a basic pH (pH of greater than 7 to 14). In some embodiments, the pH responsive hydrogel matrix can include acidic and/or basic polymers. Exemplary suitable acidic polymers include, without limitation, PAA, PMAA, poly(L-glutamic acid), polymers containing sulfonamide, and any combination thereof are the most commonly used acidic polymers for drug delivery. Exemplary suitable basic polyelectrolytes and polymers include, without limitation, poly(2-(dimethylamino) ethyl methacrylate) and poly(2-(diethylamino) ethyl methacrylate), poly(2-vinylpyridine), and biodegradable poly(β-amino ester), and any combination thereof.

In some embodiments, the injectable hydrogel is temperature responsive. In other words, the hydrogel is responsive to a specific temperature or range of temperatures. In some embodiments, the injectable hydrogel is responsive to a physiologic temperature. In some embodiments, the injectable hydrogel is responsive to a temperature in the range of 0 to about 150 degrees C. In some embodiments, the injectable hydrogel is responsive to a temperature in of about 0, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100, 100.5, 101, 101.5, 102, 102.5, 103, 103.5, 104, 104.5, 105, 105.5, 106, 106.5, 107, 107.5, 108, 108.5, 109, 109.5, 110, 110.5, 111, 111.5, 112, 112.5, 113, 113.5, 114, 114.5, 115, 115.5, 116, 116.5, 117, 117.5, 118, 118.5, 119, 119.5, 120, 120.5, 121, 121.5, 122, 122.5, 123, 123.5, 124, 124.5, 125, 125.5, 126, 126.5, 127, 127.5, 128, 128.5, 129, 129.5, 130, 130.5, 131, 131.5, 132, 132.5, 133, 133.5, 134, 134.5, 135, 135.5, 136, 136.5, 137, 137.5, 138, 138.5, 139, 139.5, 140, 140.5, 141, 141.5, 142, 142.5, 143, 143.5, 144, 144.5, 145, 145.5, 146, 146.5, 147, 147.5, 148, 148.5, 149, 149.5, or/to about 150 degrees C. or any range of values therein. In some embodiments, the injectable hydrogel is responsive to a temperature in the range of about 25 degrees C. to about 40 degrees C., about 30 degrees C. to about 40 degrees C., about 35 degrees C. to about 40 degrees C., about 35.5 degrees C. to about 40 degrees C., about 36 degrees C. to about 40 degrees C., about 36.5 degrees C. to about 40 degrees C., about 36.6 degrees C. to about 40 degrees C. about 36.7 degrees C. to about 40 degrees C., about 36.8 degrees C. to about 40 degrees C., about 36.9 degrees C. to about 40 degrees C., about 37 degrees C. to about 40 degrees C., about 37.1 degrees C. to about 40 degrees C., about 37.2 degrees C. to about 40 degrees C., about 37.3 degrees C. to about 40 degrees C., about 37.4 degrees C. to about 40 degrees C., about 37.5 degrees C. to about 40 degrees C., about 37.6 degrees C. to about 40 degrees C., about 37.7 degrees C. to about 40 degrees C., about 37.8 degrees C. to about 40 degrees C., about 37.9 degrees C. to about 40 degrees C., about 38 degrees C. to about 40 degrees C., about 38.1 degrees C. to about 40 degrees C., about 38.2 degrees C. to about 40 degrees C., about 38.3 degrees C. to about 40 degrees C., about 38.4 degrees C. to about 40 degrees, C about 38.5 degrees C. to about 40 degrees C., about 39 degrees C. to about 40 degrees C., about 39.5 degrees C. to about 40 degrees C. In some embodiments, the hydrogel is responsive to a temperature of about 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, or about 45 degrees C. In some embodiments, the hydrogel is responsive to a temperature of about 35, 35.5, 36, 36.5, 37, 37.5, 38, 38.5, 39, 39.5, or about 40 degrees C. In some embodiments, the hydrogel is responsive to a temperature of about 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9 or about 40 degrees C. In some embodiments, the hydrogel is responsive to a temperature of about 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, or about 39 degrees C.

Exemplary suitable temperature responsive polymers include, but are not limited to, poly(N-isopropylacrylamide) (PNIPAAm) and poly(N,N-diethylacrylamide) (PDEAAm). PDEAAm has a low value of LCST (a critical temperature below which the components of a solution with any composition are miscible) in the range of 25-32° C., which is near to normal body temperature. Other temperature responsive polymers are generally known in the art.

### Methods of Making the Injectable Hydrogel

In some embodiments, a thiol-Michael addition reaction, such as a thiol-Michael addition click reaction is used to generate the injectable hydrogel. Thus, in some embodiments, the injectable hydrogel is a thiol-Michael addition hydrogel. Thiol-Michael addition reactions and their use to generate various hydrogels is generally known in the art. In some embodiments, the injectable hydrogel is a reaction product of a polymer including at least one Michael acceptor and a thiol compound reacted in the presence of an aqueous base. Thus, in some embodiments, the injectable hydrogel can be formed by reacting a polymer including at least one Michael acceptor and a thiol compound in the presence of an aqueous base.

In some embodiments, at least one of the one or more polymers of the hydrogel is composed of one or more monomers that is/are a Michael acceptor. In some embodiments, the Michael acceptor is acrylate, vinyl nitrile, vinyl nitro, vinyl phosphonate, vinyl sulfonate, or a compound having an enone.

In some embodiments, the thiol compound is a multi-arm, thiol terminated polymer comprising a backbone composed of: poly(ethylene glycol), polycaprolactam, poly(propylene glycol), and poly(lactide) chains, and any water-soluble polysaccharide functionalized with 3 or more (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 1, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more) thiol groups per chain. In some embodiments, the thiol compound is a multi-arm, thiol-terminated polyethylene glycol (PEG) oligomer or ethoxylated trimethylolpropane tri-3-mercaptopropionate. In some embodiments, the thiol-terminated PEG oligomer has an average molecular weight less than about 100,000 g/mol but is greater than zero.

In some embodiments, the aqueous base is an inorganic carbonate, an inorganic bicarbonate, a buffer having a pH ranging from 7.4-14, an amine base, or any combination thereof.

In some embodiments, the aqueous base is NaHCO₃In some embodiments, the concentration of the aqueous base is about 0.1 M to about 0.25 M (e.g., about 0.1, 0.11, 0.12, 0.13, 0.14, 0.15, 0.16, 0.17, 0.18, 0.19, 0.2, 0.21, 0.22, 0.23, 0.24, or about 0.25 M).

### Kits

Any of the hydrogels, agents, hydrogel reagents (e.g., polymers, monomers, aqueous bases, and/or the like) and/or the like described herein or a combination thereof can be presented as a combination kit. As used herein, the terms “combination kit” or “kit of parts” refers to the hydrogels, agents, hydrogel reagents (e.g., polymers, monomers, aqueous bases, and/or the like) and/or the like, and any additional components that are used to package, sell, market, deliver, and/or administer the combination of elements or a single element, such as the active ingredient, contained therein. Such additional components include, but are not limited to, packaging, syringes, blister packages, bottles, electric probes, ultrasound probes, and/or the like. The separate kit components can be contained in a single package or in separate packages within the kit. Components within the kit can be configured for single use. Components within the kit can be configured for reuse and optionally for sterilization.

In some embodiments, the combination kit also includes instructions printed on or otherwise contained in a tangible medium of expression. The instructions can provide information regarding the content of the hydrogels, agents, hydrogel reagents (e.g., polymers, monomers, aqueous bases, and/or the like) and/or the like contained therein, safety information regarding the content of the hydrogels, agents, hydrogel reagents (e.g., polymers, monomers, aqueous bases, and/or the like) and/or the like contained therein, information regarding the dosages, indications for use, and/or recommended treatment regimen(s) for the hydrogels, agents, hydrogel reagents (e.g., polymers, monomers, aqueous bases, and/or the like) and/or the like contained therein. In some embodiments, the instructions can provide directions for administering the hydrogels, agents, hydrogel reagents (e.g., polymers, monomers, aqueous bases, and/or the like) and/or the like described herein or a combination thereof to a subject in need thereof. In some embodiments, the instructions provide that the subject in need thereof has a cancer, such as a solid tumor cancer. In some embodiments, the instructions provide that the subject in need thereof has a glioma. In some embodiments, the instructions provide that the subject in need thereof has a glioblastoma. In some embodiments, the instructions provide that the subject in need thereof has GBM.

### Methods of Using the Injectable Hydrogels

Described in several exemplary embodiments herein are methods of using the injectable hydrogels. In some embodiments, the hydrogel is prepared outside of a subject and then subsequently delivered to a subject, such as into a cavity of a subject. In some embodiments, a hydrogel forming solution containing all the necessary components and any agents is mixed and injected or otherwise delivered to a subject, such as to a cavity (e.g., surgical cavity, resection cavity, etc. cavity) prior to hydrogel formation. In these embodiments, as the hydrogel forms, it can conform to the exact shape of the cavity.

In some embodiments, after delivery to a subject, one or more stimuli are applied to the injectable hydrogel and/or the cavity surrounding the hydrogel. In some embodiments an external stimulus effective to stimulate migration of one or more cells to the hydrogel is applied to the hydrogel or cavity surrounding the hydrogel in the subject. In some embodiments, such an external stimulus is an energy (e.g., an electrical energy, light energy, thermal energy, magnetic energy, acoustic energy, and/or the like).

In some embodiments, a stimulus is applied to the hydrogel that causes release and/or enhances release of an agent contained in the hydrogel into the environment surrounding the hydrogel. In some embodiments, the stimulus effective to cause cell migration to the hydrogel can also be effective to trigger agent release from the hydrogel. In some embodiments, the stimulus effective to cause cell migration to the hydrogel does not trigger agent release from the hydrogel. In some embodiments where the agent released, the agent is a cell migration modulator. In some embodiments, the release can enhance and/or facilitate electrotaxis or other energy taxis (e.g., phototaxis, magnetic taxis, acoustictaxis, etc.) of cells present in the environment surrounding the hydrogel to the hydrogel (such as GSCs, CSC, GBM, or other cancer cells) for capture.

Once captured and/or retained in the hydrogel, cells can be killed, inhibited, or otherwise manipulated by one or more agents present in the hydrogel (e.g., chemotherapeutic, immunomodulator, cell growth inhibitor, gene modifying agent, and/or any other suitable agent), and/or by another stimulus applied to the cells and/or hydrogel, such as an energy (e.g., a thermal energy (e.g., heat or cold ablation), a light energy (e.g., photo ablation), e.g., acoustic energy (e.g., ultrasound ablation), or any combination thereof.

In some embodiments, the release of one or more agents, such as a cell migratory modulator, from the hydrogel creates a chemical (or biological) gradient that attracts and guides cells to the hydrogel. It can be undesirable to attract non-tumor or non-cancer cells (e.g., normal, healthy cells) to the hydrogel. In some embodiments, applying an energy (e.g., electrical (e.g., current and/or voltage), thermal (heat or cold), acoustic (e.g., ultrasound), in addition to the chemical or other gradient from the hydrogel, cancer cells, particularly cancer stem cells, can be selected for. In an example embodiment, when an electric field is applied, cells orient in the direction of the field for biased migration, with the directional bias being voltage dependent. Similar to chemotaxis, this directional sensing of the electric field does not require cell migration feedback. When a low voltage, direct current electric field is applied to cells, it acts as a cue to direct their migration, and electrotaxis occurs. This phenomenon results in cell-specific responses and does not negatively affect cell viabilities, as the sub-lethal field strength does not disrupt the cell membrane. Without being bound by theory, differential ion channel expression between healthy and cancerous (e.g., malignant) cells can be used to preferentially sensitize the cancer tumor and/or cancer cells (e.g., GBM and/or GSCs) to directed cell migration by chemotaxis and electrotaxis for selective capture into the hydrogel. In some embodiments, the orientation of the electric field and/or placement of the cathode can allow for selective chemo/electrotaxis of the cancer and cancer stem cells (e.g., GBM and/or GSCs).

In some example embodiments, the cathode is placed in/adjacent to/in proximity to the hydrogel/resection cavity and the anode near e.g., a tumor chamber such that the cells (e.g., cancer and cancer stem cells (e.g., GBM and/or GSCs)) will migrate towards and cluster the cathode. In some embodiments, the cells, such as cancer and cancer stem cells (e.g., GBM and/or GSCs) preferentially cluster at the cathode. In some example embodiments, the anode is placed in/adjacent to/in proximity to the hydrogel/resection cavity and the cathode is near e.g., a tumor chamber such that the cells (e.g., cancer and/or cancer stem cells) cluster at the anode. In some embodiments, the cells, such as cancer and cancer stem cells (e.g., GBM and/or GSCs) preferentially cluster at the anode.

Once captured, the cells can be killed, modified, or otherwise manipulated. This can occur by any suitable methodology, agent(s), and/or technique. In some embodiments, the captured and/or retained cells in the hydrogel are killed, modified, or otherwise manipulated by one or more agents in the hydrogel. In some embodiments, the captured and/or retained cells in the hydrogel are killed, modified, or otherwise manipulated by one or more stimuli being applied to the hydrogel (e.g., an energy, (e.g., an electric energy, a thermal energy, an electromagnetic or photo energy, a magnetic field, an acoustic energy, and/or the like). In some embodiments, the energy is effective to ablate the cells, such as thermal ablation, photoablation, acoustic ablation, and/or the like. In some embodiments, one or more of the agents in the hydrogel is a genetic modifying agent and when delivered to a captured cell can modify the cell to treat, modify, or kill the cell. In some embodiments, the hydrogel can be removed after capturing cells.

The hydrogels described herein can be used in a therapy for a cancer, such as a solid tumor cancer. In some embodiments, the cancer is a glioma. In some embodiments, the cancer is a glioblastoma. In some embodiments, the cancer is a GBM. In some embodiments, the hydrogel is used post-surgical resection of all or part of the solid tumor. In some embodiments, the cavity in which the hydrogel is implanted or otherwise formed is the tumor resection cavity.

The use of the hydrogels described herein can be used as a combination therapy with one or more other treatment modalities, including, resection, radiation, other systemic or local chemotherapeutics, and/or any combination thereof.

Described in certain example embodiments herein are methods, such as methods for treating a cancer (e.g., a glioma) in a subject that include injecting, into a body cavity of a subject, an injectable hydrogel as previously described and/or demonstrated in the Working Examples herein, or one or more reagents capable of forming an injectable hydrogel as previously described and/or demonstrated in the Working Examples herein into the body cavity so as to form an injected injectable hydrogel in the cavity after injection; releasing the cell migratory modulating agent from the injected injectable hydrogel so as to form a chemotaxis gradient in the environment around the hydrogel; and applying an electrical energy to the injectable hydrogel and/or body cavity of the subject so as to stimulate selective migration of one or more cancer cells and/or cancer stem cells towards the injected injectable hydrogel.

In some embodiments, the voltage of an electrical energy applied to the hydrogel to drive migration to the hydrogel is about 3 to about 500 V/m. In some embodiments, the voltage of an electrical energy applied to the hydrogel to drive migration to the hydrogel is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 to/or about 500 V/m, or any range of values therein. In some embodiments, the voltage of an electrical energy applied to the hydrogel to drive migration to the hydrogel is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, to/or 250, V/m. In some embodiments, the voltage of an electrical energy applied to the hydrogel to drive migration to the hydrogel is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 V/m.

The electrical energy, no matter if used to drive migration or kill or otherwise modify captured cells can be delivered by one or more probes. In some embodiments, a cathode probe or cathode portion of a probe is positioned in, adjacent to, or in proximity to the injected injectable hydrogel. This, as previously described, can facilitate selective migration of cells (e.g., cancer and/or cancer stem cells (e.g., GBMs and/or GSCs)) towards the hydrogel and to cluster at the cathode and the hydrogel. In some embodiments, an anode probe or anode portion of a probe is positioned in, adjacent to, or in proximity to the injected injectable hydrogel. This, as previously described, can facilitate selective migration of cells (e.g., cancer and/or cancer stem cells (e.g., GBMs and/or GSCs)) towards the hydrogel and to cluster at the anode and the hydrogel.

In some embodiments, the same probes, in the same or different placement or configuration as for electrotaxis, are used to provide a therapeutic electrical energy (such as to generate heat for thermal ablation or provide an electrical current for electrical ablation). In some embodiments the one or more probes or other energy sources (such as those delivering therapeutic ultrasound or light) are placed in operable proximity to the injected injectable hydrogel and can deliver a stimulus. Such stimuli can operate to, for example, cause release of agents from the hydrogel and/or kill or otherwise modify cell captured within the hydrogel.

In some embodiments, the method further includes exposing the injected injectable hydrogel to an external stimulus to the hydrogel after a period of time sufficient to capture one or more cells in the injected injectable hydrogel. In some embodiments, a period of time sufficient to capture one or more cells in the injected injectable hydrogel is or ranges from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or 60 seconds, minutes, hours, days, or weeks.

In some embodiments, the external stimulus is an energy capable of ablating one or more cells captured in the injected injectable hydrogel. In some embodiments, the energy is an electric energy, a light energy, a magnetic energy, a thermal energy, an acoustic energy, a chemical energy, a biochemical energy, a radiation energy, or a combination thereof. In some embodiments, the external stimulus is an electric energy or an acoustic energy. In some embodiments, the acoustic energy is ultrasound.

The probes or other stimulus delivery devices can be coupled to, placed in operable proximity to, or otherwise operably coupled to the hydrogel. In some embodiments the one or more probes or other energy sources (such as those delivering therapeutic ultrasound or light) are placed in operable proximity to the injected injectable hydrogel.

In some embodiments, the one or more cancer cells and/or cancer stem cells comprises a cancer cell, an immune cell, a tumor microenvironment cell, a stem cell, a circulating cell, or any combination thereof. In some embodiments, the one or more cancer cells and/or cancer stem cells is a cancer stem cell; a circulating cancer cell; a migrating cancer cell; a disseminating cancer cell; a glioma cell, tumor initiating cell, cancer-associated fibroblast; or any combination thereof. In some embodiments, the one or more cancer cells and/or cancer stem cells originates from the body cavity microenvironment.

In some embodiments, the external stimulus is capable of damaging, killing or otherwise modifying one or more cells attracted to, in immediate proximity of, contained in the injected injectable hydrogel, or any combination thereof killing or ablating one or more cells attracted to, in immediate proximity of, contained in the injected injectable hydrogel; or any combination thereof or any combination thereof.

In some embodiments, the body cavity is a surgical cavity. In some embodiments, the surgical cavity is formed from removing a tumor from the body of the subject. In some embodiments, the body cavity is in the brain of the subject. In some embodiments, the tumor is a glioma.

The methods described herein can be used to treat a cancer in a subject. In some embodiments, the cancer is a glioblastoma. In some embodiments the glioblastoma is glioblastoma multiforme. In some embodiments, the method can further include imaging the injected injectable hydrogel after injecting into a subject. In some embodiments, the hydrogel can contain one or more imaging agents.

In some embodiments, the method further includes releasing one or more agents included in the injectable hydrogel over a period of time. In some embodiments, the one or more agents released is the cell migratory modulating agent. In some embodiments, the one or more agents released is not one of the cell migratory modulating agent. In some embodiments, both the cell migration modulating agent and at least one other agent present in the injected injectable hydrogel is released. Where agent(s) are released, the individual release profile and/or kinetics for each agent can be the same or different. Release of an agent from the hydrogel can range from 1-1,000 pg, pL, pM, ng, nL, nM, μg, μL, μM, per second, minute, hour, or day.

In some embodiments, the method includes removing the injected hydrogel after one or more cells are collected therein.

In some embodiments, the method includes delivering to a subject one or more chemotherapeutics, therapeutic radiation, or both.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

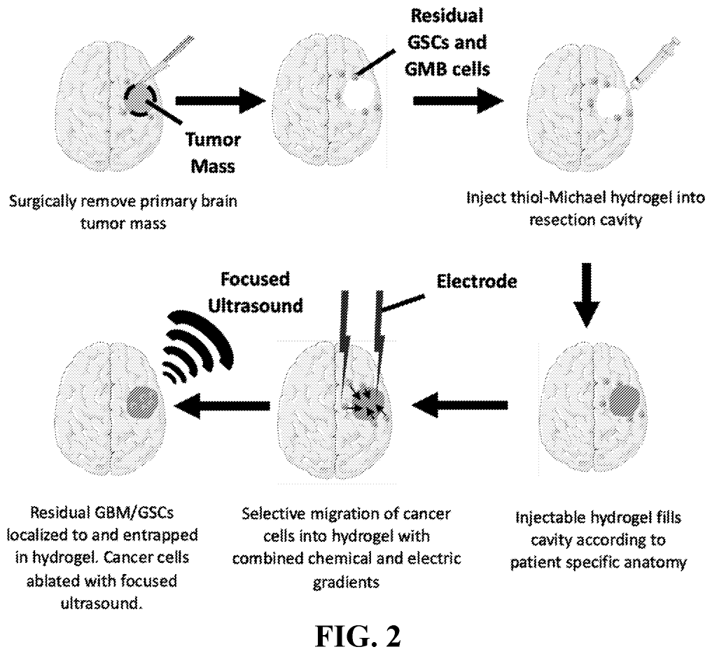

Described in this Example is an injectable hydrogel which can capture residual glioblastoma (GBM) cells and glioma cancer stem cells (GSCs). After surgical resection of the tumor mass, the hydrogel can be injected into the cavity, where the physiologic temperature can allow it to solidify. A combination of chemical and electric stimuli can direct the selective migration of malignant GBM and GSCs into a scaffold for subsequent entrapment of the cells into a localized region. The slow release of chemokines from the hydrogel can generate a chemotactic gradient to attract migratory cells toward the scaffold. A direct current electric field generated by minimally invasive microelectrodes placed in the hydrogel and the surrounding brain tissue can selectively direct the GBM and GSCs to migrate to the scaffold, and focal adhesion to the scaffold can allow the cells to pass through the hydrogel mesh to become entrapped inside. Furthermore, focused ultrasound can be used to non-invasively ablate the malignant cells once they are localized in the hydrogel scaffold. A schematic representation of this novel treatment method is outlined by FIG. 2.

The platform can include a polyethylene glycol (PEG) based hydrogel synthesized using a thiol-Michael addition click reaction with a mild and biocompatible base (NaHCO₃) catalyzing the addition of a thiolate (trithiol thiocure) into an electron deficient alkene, which in this case is polyethylene glycol diacrylate (PEGDA), in a 1:1 stoichiometric ratio¹. After maximum surgical resection of the primary tumor mass, the hydrogel solution can be injected into the resection cavity with a syringe, where it can solidify and form into a gel.

Cell migration can be directed to distal sites through chemical gradients by utilizing chemokines to attract cells to a particular location². Chemokines are chemotactic cytokines, a small class of proteins, which can mediate chemoattraction of nearby responsive cells through chemotaxis. One of the most widely studied and established chemokines for GBM migration is the stromal cell-derived factor CXCL12³, while the chemokine CX3CL1 has been reported to mediate GSC migration in primary tumors⁴. These two chemokines can be mixed with the hydrogel solution, so that upon hydrogel solidification in the resection cavity, the chemokines can be loaded inside. The slow release of the chemokines from the hydrogel can generate a concentration gradient to recruit and attract both GBM and GSCs and direct their migration toward the scaffold.

While chemokines can attract cancer cells, they also influence the migration of any nearby cells that may potentially respond to the chemical cue. Any cells in the brain with a high potential to migrate can be affected by these chemical cues⁵. Therefore, a strategy to influence the migration of GBM and GSCs selectively for cell specificity to these attracting cues was developed, without affecting normal glial cells in the brain, in order to entrap only the malignant cells in the scaffold. Such a strategy can ensure that the normal brain cells and the tissue environment in the brain are preserved during therapy. When an electric field is applied, cells orient in the direction of the field for biased migration, with the directional bias being voltage dependent⁶. Similar to chemotaxis, this directional sensing of the electric field does not require cell migration feedback⁷. When a low voltage, direct current electric field is applied to cells and acts as a cue to direct their migration, electrotaxis occurs⁸. This phenomenon results in cell-specific responses and does not negatively affect cell viabilities, as the sub-lethal field strength does not disrupt the cell membrane⁹.

Tsai and colleagues recently demonstrated that voltage-gated calcium and potassium ion channels, as well as acid-sensing sodium channels, mediate the electrotaxis of GBM cells¹⁰. Without being bound by theory, electrotaxis is another form of chemotaxis in which extracellular molecules can undergo electrophoresis and redistribute to form a chemical gradient⁸. Accumulating research in the field has implicated that an overexpression of ion channels in GBM cells regulate the cytoplasmic volume and drive the migration of these cells through hydrodynamic volume changes arising from ionic gradients¹¹. Results from a study conducted by Lyon and colleagues show that application of a direct current electric field can cluster dispersed GBM tumor aggregates together in 3D models. Previous research indicated that both a GBM cell line 8 and GBM tumor initiating cells⁶ oriented and migrated in a cathode-biased direction when direct current electric field was applied in a 3D environment. The compositions and methods described in this example can exploit the principle that there is differential ion channel expression between malignant and non-malignant brain cells and use it as a physical hallmark to preferentially sensitize GBM and GSCs to make them more susceptible to directed cell migration by chemotaxis and electrotaxis for selective capture into the scaffold. Minimally invasive microneedle electrodes can be used to generate an electric field across the hydrogel in situ. The anode can be placed near the tumor chamber of the platform, while the cathode can be placed near the tumor resection cavity chamber to ensure that the electrotaxis helps to direct cell migration of the tumor cells toward the thiol-Michael hydrogel.

Upon localization of the malignant cells into the hydrogel, the scaffold can remain as an implant in the brain of the glioblastoma patient. The continuous release of chemokines over a period of time can help to entrap more cancer cells. Ablation of the malignant cells can be performed using focused ultrasound, in order to avoid complications which may arise from the surgical removal of the hydrogel, although surgical removal of the hydrogel can be used and may be appropriate in some instances as will be understood by a medical practitioner. Specifically, histotripsy can be applied, which is a noninvasive, non-thermal focused ultrasound therapy utilizing high amplitude and short duration ultrasound pulses applied via an extracorporeal ultrasound transducer ¹². The rapid expansion and collapse of bubbles can lead to localized strains and stresses to mechanically fractionate the cells in the hydrogel ¹². The histotripsy parameters, such as pulsed repetition frequency, amplitude, and cavitation thresholds can be tailored to target the cells entrapped in the hydrogel scaffold. Furthermore, MRI and CT imaging can be coupled with the focused ultrasound ablation in order to guide histotripsy to result in targeted and well-defined lesion areas in the hydrogel scaffold ^{13,14}.

Without being bound by theory, injectable hydrogel and methods of uses described in this Example can have advantages of current cancer therapy approaches, particularly those for glioblastomas and other cancers whose treatment efficacy and long-term survival and recurrence rates suffer from a residual cancer cell population.

While previous research to date which have developed scaffolds to entrap cancer cells have been hydrogels which undergo gelation ex vivo for implantation, the instant biomaterial scaffold can be a viscous solution ex vivo so that it can be injected in vivo with a syringe to undergo the solution to gel transition in situ under physiologic conditions. Since the starting reagents can be sterilized prior to injection and hydrogel solidification, there can be no need to sterilize preformed gels using gamma radiation prior to implantation. Injectable hydrogels undergo rapid gelation without UV light or reaction initiators¹⁵, rendering them with highly biocompatible properties. Research conducted by Peach and colleagues indicated that the polymerization reaction occurs with minimum heat production with only a five degree Celsius increase before and after hydrogel solidification¹⁶, thereby demonstrating that the in situ hydrogel gelation process will not cause patient discomfort¹⁷. Furthermore, Applicant's hydrogel platform can be able to naturally conform to the patient specific anatomy by filling the tumor resection cavity uniformly, regardless of its irregular shape, to result in a homogenous hydrogel¹⁸.

Biomaterial implants can trigger an immune response¹⁹, but the post-surgery reverse migration theory postulates that an immune response can help enhance GSC migration into the cavity²⁰. Immunomodulation has previously been demonstrated to recruit metastatic cancer cells into a pre-metastatic niche scaffold²¹. Post-surgery, patients typically have to wait for a period of time to allow the resection cavity to heal before chemoradiotherapy is administered²². Hence, this engineered injectable hydrogel can be used as adjuvant therapy to fill the gap between surgery and chemoradiotherapy to capture the residual GBM and GSCs responsible for tumor recurrence and complement therapies which aim to shrink tumor masses.

PEG-based biomaterials are particularly advantageous, since their biocompatibility enables them to be approved for in vivo implantation by the FDA¹. Compared to purely PEGDA-based hydrogels, the addition of a thiol group allows the network to be less brittle and more compliant¹⁵. Moon and colleagues demonstrated the suitability of this injectable hydrogel for clinical applications, as according to their research, the reaction was very rapid and took less than two minutes under mild and biofriendly conditions¹. Furthermore, computed tomography (CT) studies revealed the hydrogel can be detected under CT imaging for image guided treatment¹. This hydrogel can be synthesized from commercially available and inexpensive oligomeric starting materials¹. Since parameters such as the initial water content and base concentration can be varied to change the injectable hydrogel properties upon formation¹, this thiol-Michael injectable hydrogel is highly tunable as the base biomaterial platform to engineer the scaffold which can capture residual GBM cells in the tumor resection cavity. Furthermore, a high water content increases the visibility of the hydrogel to imaging modalities such as MRI and is an important consideration when imaging is needed for precise and targeted treatment to eradicate the concentrated cells in the hydrogel¹⁹.

Autier and colleagues had developed a bacterial cellulose (BC) based hydrogel scaffold which can release chemoattractants to generate a chemical gradient to attract and immobilize GBM cells with surface adhesion¹⁹. Since the GBM cells are not entrapped within the scaffold itself, there is risk of tumor cell escape and invasion into the brain parenchyma. In a separate study, Jain and colleagues engineered nanofibers to guide GBM cells away from the primary tumor mass to an extracortical location. This extracortical location contains a cyclopamine drug-conjugated, collagen-based hydrogel to induce apoptosis in both GBM and GSCs, without toxicity to normal brain cells ²³. However, if the polymeric nanofiber conduit transporting the cells is compromised, there is a risk of infiltration of the migrating tumor cells into healthy brain tissue. In both of these cases, GBM tumor cell escape from the scaffolds can result in their invasion and migration along white matter tracts and blood vessels to result in secondary tumor sites in the brain. This treatment plan can mitigate this risk, since the GBM cells can be entrapped within the hydrogel scaffold itself. It has been reported that Michael addition reactions are ideal for cell and protein encapsulation in hydrogels ²⁴.

The BC membrane developed by Autier et al. is not degradable, since the in vivo studies with rat models indicated that the scaffolds are stable up to even 12 months upon implantation ¹⁹. As such, the authors observed a chronic inflammatory response with altered neutrophils, multinucleated giant cells, fibroblasts with collagen fibers and neovascularization in their in vivo experiments. While cellulase can be administered to degrade cellulose based hydrogels ², this additionionL step in the treatment plan can lead to complications. Furthermore, bacterial cellulose can lead to residual bacterial biomolecules in the biomaterial scaffold¹⁹ remaining even after sterilization. In contrast, the hydrophilic properties of PEG result in the dominant mechanism for its degradation through bulk degradation, by the hydrolysis of the ester bonds present in the hydrogel matrix¹⁵. Hence, upon FUS ablation of the entrapped cancer cells, invasive surgical methods will not be required to remove the hydrogel scaffold, since it can naturally degrade with time. Hydrogel-based biomaterials possess the risk of soluble components in the network leaching into surrounding tissues, since network defects which result in leached reagents can potentially harm surrounding healthy tissue and lead to reduction in optimal performance of hydrogels¹⁵. However, thiol-Michael hydrogels were previously demonstrated to possess high gel fractions ranging from 83.6% to 97.2%, thereby indicating high conversions for the reaction, while any unreacted oligomers failed to incite significant toxicities¹.

The mechanical properties of the thiol-Michael hydrogel are compatible with brain tissue. For example, the stiffness of brain tissue is approximately 1 kPa²⁵, and since the storage modulus of thiol-Michael hydrogels are in the kPa magnitude, but higher than 1 kPa¹, the hydrogel can possess a high enough modulus to maintain its structural integrity under tissue forces, while simultaneously being able to distend brain tissue during polymerization and swelling¹⁶. In the treatment plan, PEGDA oligomers were used with a molecular weight of about 575 Da to synthesize the hydrogel scaffold. Lower chain PEGDA oligomers lead to less swelling of hydrogel, but lower chain oligomers also lead to insolubility in water¹. Therefore, median chain length of PEGDA of about 575 Da was used.

### Chemical and Electric Stimuli Directed Cancer Cell Migration

In their development of a fake metastatic niche to trap metastatic cancer cells, Giarra and colleagues observed that while certain hydrogel formulations loaded with chemokines were effective at inducing cell migration toward the hydrogel, they were not necessarily effective at capturing the cells and trapping them². Autier and colleagues had performed organotypic brain slice invasion assays to assess the GBM cell behavior at a distance from their hydrogel scaffold. The researchers discovered that the rapid release of chemokines from their hydrogel scaffold was unable to establish a high enough concentration gradient for the GBM cells to sense the chemical cue for chemotaxis across long distances to migrate toward their hydrogel¹⁹. The authors purport that chemoattractants may only be able to diffuse distances between 1-5 mm through brain tissue. In order to address these issues, the instant strategy can use both chemical and electric stimuli to guide the cancer cells to the scaffold.

Electrotaxis can allow the migration of the malignant cells across longer distances toward the attracting scaffold. Non-contact force directs the cell motility in electrotaxis, and can control tumor dispersion⁸. Studies demonstrate the ability of direct current electric fields to cluster or move dispersed tumor aggregates in tumor models intentionally as a way to coalesce dispersed brain tumor aggregates⁸. In contrast to the cell trap developed by Autier and colleagues targeting GBM cells only, the instant hydrogel platform can entrap both GBM and GSC populations. Since the voltage parameters for the direct current in electrotaxis is highly tunable to influence cell migration⁶, selective migration of both cell types can be directed toward the scaffold, without inducing cell migration in normal brain cells.

Moreover, utilizing electrotaxis also presents an opportunity for patient specific tailored treatment. Fundamental heterogeneity in GBM cells contribute to recurrence and therapeutic resistance after therapy²⁶. Research conducted by Tsai and colleagues had demonstrated that within different human glioblastoma cell lines, there are different biases of cell migration direction as well as speeds²⁶, indicating that electrotaxis directed cell migration may be patient specific. Hence, an extracorporeal platform can be used to assess the electrotactic responses to specific voltage parameters in patient specific GBM samples prior to treatment for a personalized approach to tailoring the injectable hydrogel cell entrapment treatment.

Non-Invasive Cancer Cell Ablation with Focused Ultrasound

Although not implemented or studied, Autier and colleagues had proposed that after attracting and immobilizing GBM cells on their scaffold, the destruction of the trapped cells can be facilitated by stereotactic radiosurgery, where a high dose of radiation will be delivered to specific location in order to minimize damage to surrounding normal brain tissue ¹⁹. However, such a strategy will fail to eliminate GBM recurrence, as cancer stem cells are resistant to radiation²³. Paelez and colleagues had recently developed a biomaterial scaffold to attract metastatic breast cancer cells and thermally ablate these infiltrating cells by applying non-invasive focal hyperthermia²⁷. The researchers incorporated metal disks into their scaffold to generate heat through electromagnetic induction with an external and extracorporeal magnetic field. However, the authors noted an uneven distribution of the heat source in their scaffold led to temperature gradients which were unable to uniformly destroy the trapped cancer cells. Animal model studies with mice indicated that applying this focal treatment led to tissue necrosis in tissues surrounding the scaffolds.

Using histotripsy as the non-invasive ablation technique for eliminating the GBM cells can address these issues. Since histotripsy fractionates cells mechanically and non-thermally, the lack of heat dissipation¹² can ensure that heat diffusion does not lead to uncontrolled lesion borders and necrotic tissues. Additionally, histotripsy can be administered rapidly with short durations of pulses at low duty cycles less than 0.01%, which also decreases tissue heating in the focused ultrasound beam pathway¹². The resulting acellular debris can ensure that the ablated cells will not further harm the brain microenvironment¹². The short durations of pulses also allow small focal volumes and precisely targeted and controlled lesion areas with no damage to nearby tissues¹². Previous studies have shown that histotripsy can control lesions within millimeter accuracies through the skull²⁸ without requiring aberration corrections²⁹.

### Applications of the Hydrogel Platform

The treatment plan outlined herein is a strategy to reduce tumor recurrence for GBM patients by eradicating not only residual GBM cells, but also the highly resistant GSC populations responsible for initiating new tumors. This combinatorial approach utilizes the strengths of both chemical and physical stimuli to localize residual GBM and GSCs, thereby circumventing the need for employing unreliable biomarkers to target GBM and GSC populations separately. The novel treatment can help shrink tumor masses, reduce recurrence, and lead to better prognoses for GBM patients. Post-surgery, patients typically have to wait for a period of time to allow the resection cavity to heal before chemoradiotherapy is administered²². Hence, this engineered injectable hydrogel can be used as adjuvant therapy to fill the gap between surgery and chemoradiotherapy to capture the residual GBM and GSCs responsible for tumor recurrence and complement therapies which aim to shrink tumor masses.

### References for Example 1

1 Moon, N. G., Pekkanen, A. M., Long, T. E., Showalter, T. N. & Libby, B. Thiol-Michael ‘click’ hydrogels as an imagable packing material for cancer therapy. Polymer 125, 66-75, doi:10.1016/j.polymer.2017.07.078 (2017).
2 Giarra, S. et al. Engineering of thermoresponsive gels as a fake metastatic niche. Carbohydr Polym 191, 112-118, doi:10.1016/j.carbpol.2018.03.016 (2018).
3 Clavreul, A. et al. Glioblastoma-associated stromal cells (GASCs) from histologically normal surgical margins have a myofibroblast phenotype and angiogenic properties. The Journal of pathology 233, 74-88, doi:10.1002/path.4332 (2014).
4 Locatelli, M. et al. Human glioma tumors express high levels of the chemokine receptor CX3CR1. European cytokine network 21, 27-33, doi:10.1684/ecn.2009.0184 (2010).
5 van der Sanden, B. et al. Translation of the ecological trap concept to glioma therapy: the cancer cell trap concept. Future oncology (London, England) 9, 817-824, doi:10.2217/fon.13.30 (2013).
6 Huang, Y. J. et al. Cellular microenvironment modulates the galvanotaxis of brain tumor initiating cells. Scientific reports 6, 21583, doi:10.1038/srep21583 (2016).
7 Wang, M. J., Artemenko, Y., Cai, W. J., Iglesias, P. A. & Devreotes, P. N. The directional response of chemotactic cells depends on a balance between cytoskeletal architecture and the external gradient. Cell reports 9, 1110-1121, doi:10.1016/j.celrep.2014.09.047 (2014).
8 Lyon, J. G., Carroll, S. L., Mokarram, N. & Bellamkonda, R. V. Electrotaxis of Glioblastoma and Medulloblastoma Spheroidal Aggregates. Scientific reports 9, 5309, doi:10.1038/s41598-019-41505-6 (2019).
9 Polk, C. Handbook of biological effects of electromagnetic fields. 103-148 (CRC Press, 1995).
10 Tsai, H.-F., Ijspeert, C. & Shen, A. Voltage-gated ion channels mediate the electrotaxis of glioblastoma cells in a hybrid PMMA/PDMS microdevice. (2020).
11 Cuddapah, V. A. & Sontheimer, H. Ion channels and transporters [corrected] in cancer. 2. Ion channels and the control of cancer cell migration. Am J Physiol Cell Physiol 301, C541-549, doi:10.1152/ajpcell.00102.2011 (2011).
12 Sukovich, J. R. et al. In vivo histotripsy brain treatment. J Neurosurg, 1-8, doi:10.3171/2018.4.JNS172652 (2018).
13 Marquet, F. et al. Non-invasive transcranial ultrasound therapy based on a 3D CT scan: protocol validation and in vitro results. Physics in medicine and biology 54, 2597-2613, doi:10.1088/0031-9155/54/9/001 (2009).
14 Pernot, M., Aubry, J. F., Tanter, M., Thomas, J. L. & Fink, M. High power transcranial beam steering for ultrasonic brain therapy. Physics in medicine and biology 48, 2577-2589, doi:10.1088/0031-9155/48/16/301 (2003).
15 Pritchard, C. D. et al. An injectable thiol-acrylate poly(ethylene glycol) hydrogel for sustained release of methylprednisolone sodium succinate. Biomaterials 32, 587-597, doi:10.1016/j.biomaterials.2010.08.106 (2011).
16 Peach, M. S. et al. Development and preclinical testing of a novel biodegradable hydrogel vaginal packing technology for gynecologic high-dose-rate brachytherapy. J Contemp Brachytherapy 10, 306-314, doi: 10.5114/jcb.2018.77952 (2018).
17 Lindstedt, F., Lonsdorf, T. B., Schalling, M., Kosek, E. & Ingvar, M. Perception of thermal pain and the thermal grill illusion is associated with polymorphisms in the serotonin transporter gene. PloS one 6, e17752, doi:10.1371/journal.pone.0017752 (2011).
18 Puente, P. et al. Injectable Hydrogels for Localized Chemotherapy and Radiotherapy in Brain Tumors. Journal of pharmaceutical sciences 107, 922-933, doi:10.1016/j.xphs.2017.10.042 (2018).
19 Autier, L. et al. A new glioblastoma cell trap for implantation after surgical resection. Acta biomaterialia 84, 268-279, doi:10.1016/j.actbio.2018.11.027 (2019).
20 Jabbour, W. & Wion, D. EXTENT OF RESECTION AND SURVIVAL IN GLIOBLASTOMA MULTIFORME. JAMA Oncol. 2, 1509, doi:10.1227/01.neu.0000317304.31579.17 (2016).
21 Azarin, S. M. et al. In vivo capture and label-free detection of early metastatic cells. Nature communications 6, 8094, doi:10.1038/ncomms9094 (2015).
22 Zhao, M. et al. Post-resection treatment of glioblastoma with an injectable nanomedicine-loaded photopolymerizable hydrogel induces long-term survival. International journal of pharmaceutics 548, 522-529, doi:10.1016/j.ijpharm.2018.07.033 (2018).
23 Jain, A. et al. Guiding intracortical brain tumour cells to an extracortical cytotoxic hydrogel using aligned polymeric nanofibres. Nature materials 13, 308-316, doi:10.1038/nmat3878 (2014).
24 Shah, K., Vasileva, D., Karadaghy, A. & Zustiak, S. P. Development and characterization of polyethylene glycol-carbon nanotube hydrogel composite. J Mater Chem B 3, 7950-7962, doi:10.1039/c5tb01047k (2015).
25 Hasanzadeh, E. et al. Preparation of fibrin gel scaffolds containing MWCNT/PU nanofibers for neural tissue engineering. J Biomed Mater Res A 107, 802-814, doi:10.1002/jbm.a.36596 (2019).
26 Tsai, H.-F., Ijspeert, C. & Shen, A. Q., doi:10.1101/2020.02.14.948638 (2020).
27 Pelaez, F. et al. Biomaterial scaffolds for non-invasive focal hyperthermia as a potential tool to ablate metastatic cancer cells. Biomaterials 166, 27-37, doi:10.1016/j.biomaterials.2018.02.048 (2018).
28 Kim, Y., Hall, T. L., Xu, Z. & Cain, C. A. Transcranial histotripsy therapy: a feasibility study. IEEE Transactions on Ultrasonics, Ferroelectrics, and Frequency Control 61, 582-593, doi:10.1109/TUFFC.2014.2947 (2014).
29 Sukovich, J. et al. Targeted Lesion Generation Through the Skull Without Aberration Correction Using Histotripsy. IEEE Trans Ultrason Ferroelectr Freq Control 63, 671-682, doi:10.1109/tuffc.2016.2531504 (2016).

### Example 2

Glioblastoma multiforme (GBM) is the most aggressive type of primary brain tumor. Its infiltrative capacity allows GBM to utilize a variety of strategies to diffuse to surrounding brain tissues (FIG. 1A). Although patients undergo treatments comprising “search and destroy” strategies such as surgery, chemotherapy, and radiotherapy to eradicate GBM, the glioma cancer stem cell (GSC) population resistant to therapy remain and lead to tumor recurrences^{1,2} (FIG. 1B).

Recent initiatives have developed an “attract to kill” strategy by guiding GBM cell migration into scaffolds for entrapment^{5,6,7}. However, these scaffolds do not target GSCs. A polyethylene glycol (PEG) based injectable hydrogel with a thiol-Michael click reaction can conform to patient specific anatomical cavities⁸ and the initial water content and catalyst base concentration can be tailored to influence hydrogel properties⁹. As described here, post-surgery, this platform can capture and localize residual GBM/GSCs in the tumor resection cavity with a combination of chemotaxis and electrotaxis. The tumor cells can then be ablated with a non-invasive technology such as focused ultrasound. A schematic of this treatment approach is shown in FIG. 2.

This Example describes a thiol-Michael hydrogel formulation with a specific base concentration and water content combination that enhances hydrogel properties for capturing/retaining GSCs and GBM cells. Nine hydrogel formulations containing NaHCO₃ base concentrations at three concentrations (0.1 M, 0.175 M, and 0.25 M) and initial water content by weight at three wt percents (wt %) (25 wt %, 50 wt %, and 75 wt %) were synthesized. Characterization studies to elucidate parameter-property relationships and determine the most optimal formulation for the GBM cell trap application were conducted.

### Hydrogel Synthesis

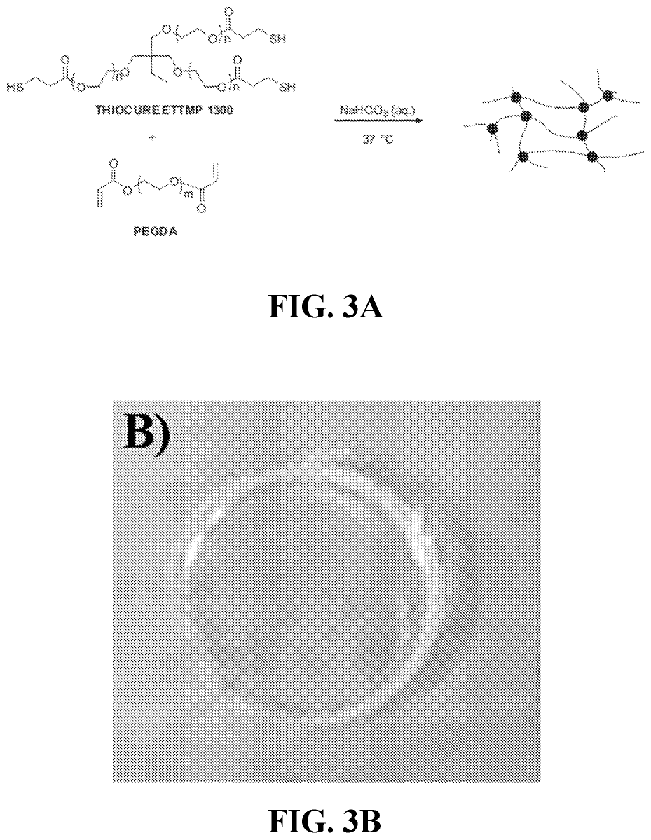

Hydrogel synthesis included the addition of the NaHCO₃ base to PEGDA and mixing by vortex to obtain a uniform solution. Polythiols (Thiocure polythiols) were injected into this mixture, which was further mixed via stirring using a stir rod for the final mixing step in order to mimic its preparation in the clinical setting. See FIGS. 3A-3B.

### Swelling Studies

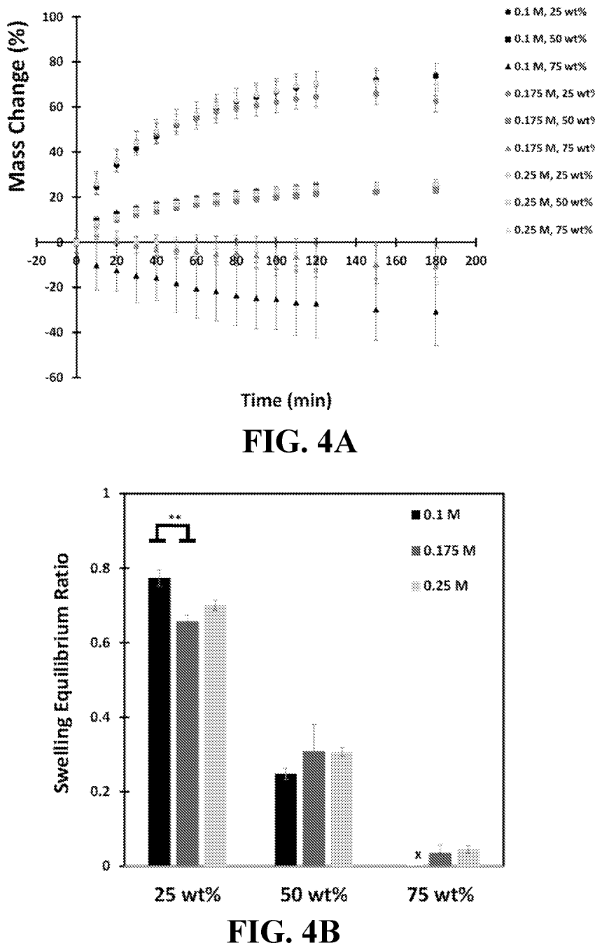

Fluid diffusion into hydrogels can result in swelling and potentially affect the polymer network within the hydrogel. Excessive swelling can be damaging to neural cells. Swelling studies were performed to determine the swelling characteristics of the various hydrogel formulations. Results are shown in FIGS. 4A-4B. These are further discussed in Example 3.

### Rheology Studies

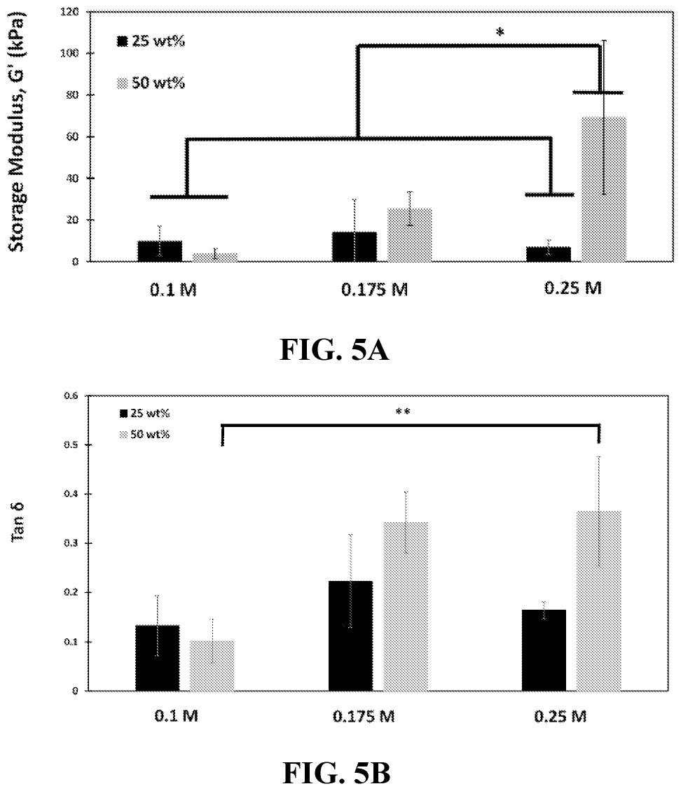

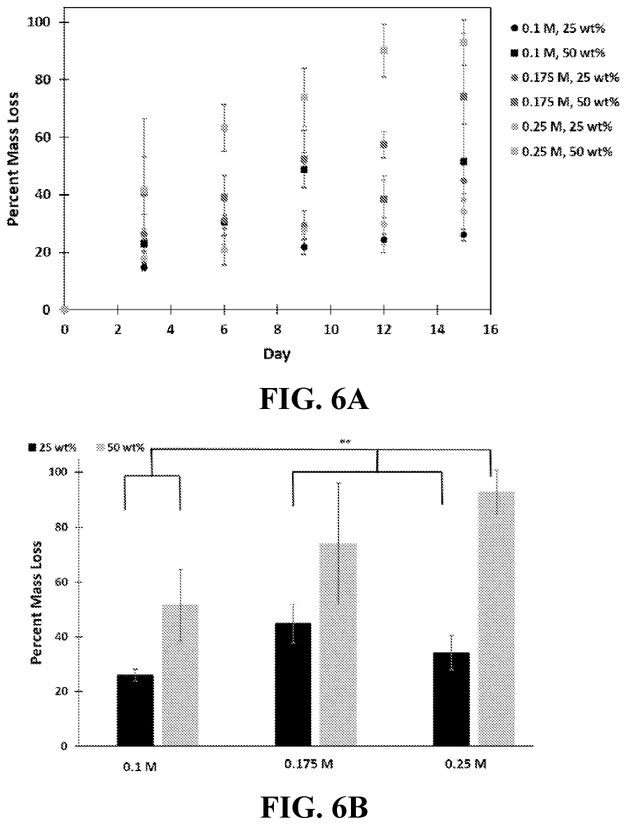

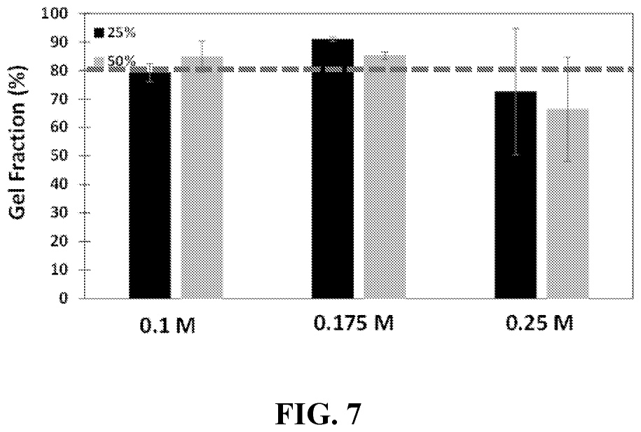

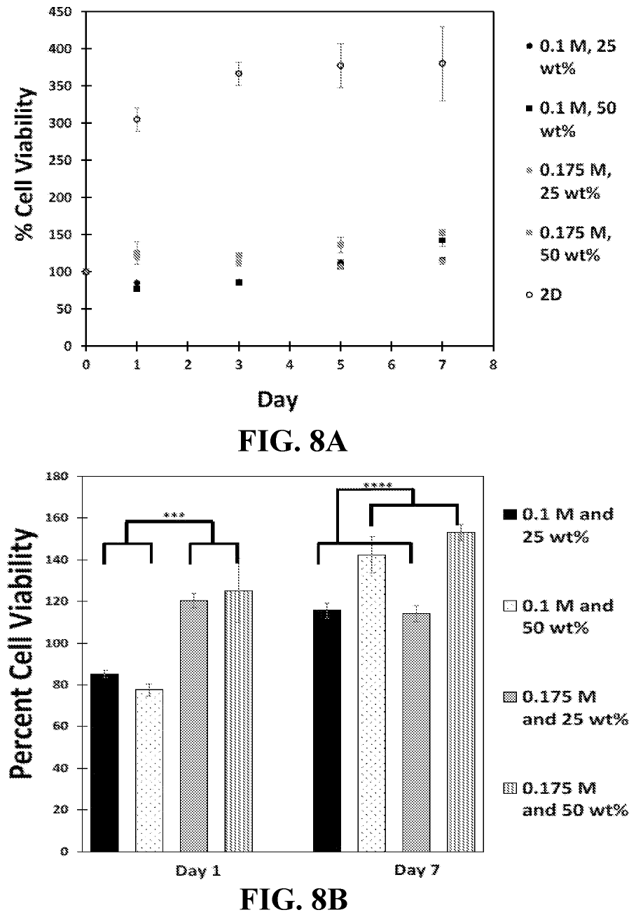

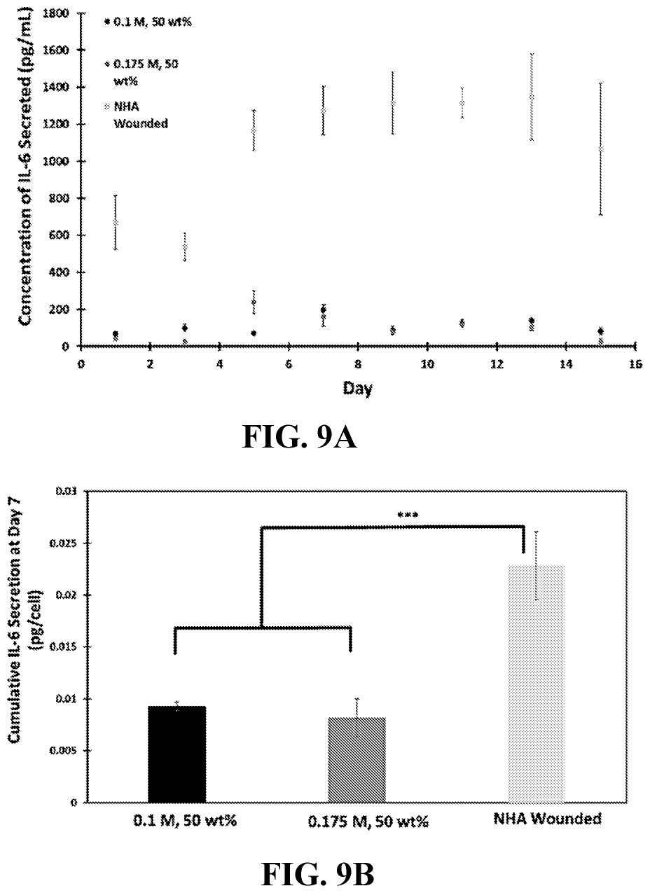

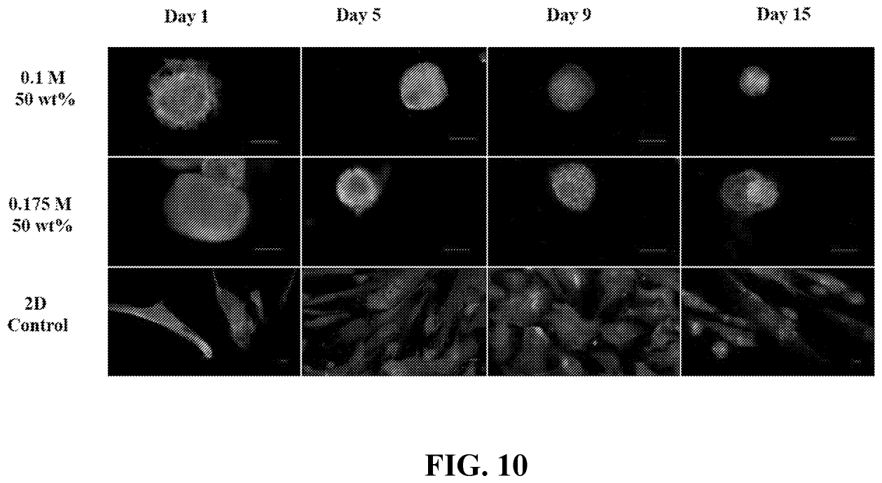

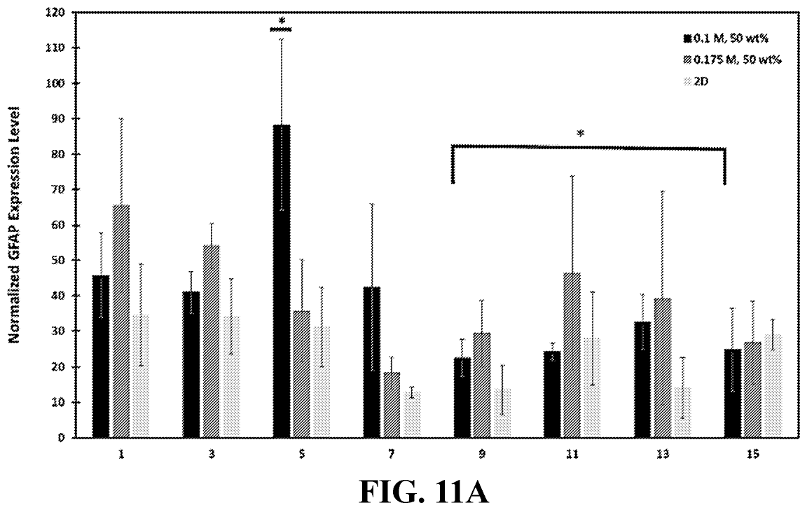

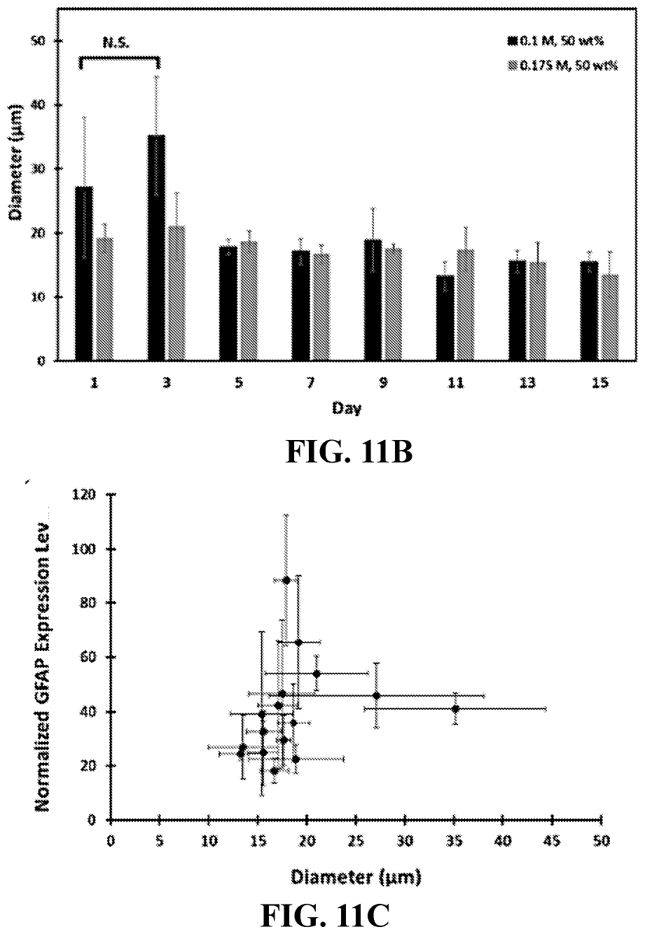

Rheology studies were performed on the hydrogel formulations to determine their ability to withstand the elevated intracranial pressure of brain tumors. The relative hydrogel formulation stiffnesses can also lend insight into rates of cell migration into the hydrogel, since a lower modulus can modulate the mesh sizes to allow cells to travel inside. Results are shown in FIGS. 5A-5B. These are further discussed in Example 3.

### Discussion

Swelling studies demonstrated that a hydrogel formulation with 75 wt % resulted in accelerated swelling, equilibrium, and disintegration of a hydrogel. Without being bound by theory, this was due to poor crosslinking and can indicate its unsustainability for capturing and retaining GSCs and/or GBM cells. As such, the hydrogel should have a wt % water content of less than 75%. All three formulations containing a 75 wt % water content were not used for further evaluation.

Rheology results indicated that the initial water content had a more significant effect on the hydrogel moduli compared to the base concentration. The hydrogel formulation with 0.25 M NaHCO₃ base and 50 wt % initial water content resulted in the hydrogel with the highest storage and loss moduli. A higher standard deviation in the moduli was observed for increasing moduli.

Rheology data also indicated the storage moduli of the hydrogels ranged from about 3 kPa to 100 kPa and not only conform to brain tissue stiffness associated with GBM compression stiffening, but also conform to the kPa range of substrate stiffnesses in which GBM cells become mechanoresponsive, thereby allowing these substrates to influence their function and motility¹⁰.

### References for Example 2

1. Singh, S. K. et al. Identification of human brain tumour initiating cells. Nature 432, 396-401, doi:10.1038/nature03128 (2004).
2. Sun, T. et al. Targeting transferrin receptor delivery of temozolomide for a potential glioma stem cell-mediated therapy. Oncotarget 8, 74451-74465, doi:10.18632/oncotarget.20165 (2017).
3. Das, S., Srikanth, M., & Kessler, J. A. (2008). Cancer stem cells and glioma. Nat Clin Pract Neurol, 4(8), 427-435. doi:10.1038/ncpneuro0862.
4. Seano, G., & Jain, R. K. (2020). Vessel co-option in glioblastoma: emerging insights and opportunities. Angiogenesis, 23(1), 9-16. doi:10.1007/s10456-019-09691-z
5. Autier, L. et al. A new glioblastoma cell trap for implantation after surgical resection. Acta biomaterialia 84, 268-279, doi:10.1016/j.actbio.2018.11.027 (2019).
6. van der Sanden, B. et al. Translation of the ecological trap concept to glioma therapy: the cancer cell trap concept. Future oncology (London, England) 9, 817-824, doi:10.2217/fon.13.30 (2013).
7. Jain, A. et al. Guiding intracortical brain tumour cells to an extracortical cytotoxic hydrogel using aligned polymeric nanofibres. Nature materials. 13, 308-316, doi:10.1038/nmat3878 (2014).
8. Puente, P. et al. Injectable Hydrogels for Localized Chemotherapy and Radiotherapy in Brain Tumors. Journal of pharmaceutical sciences 107, 922-933. doi:10.1016/j.xphs.2017.10.042 (2018).
9. Moon, N. G., Pekkanen, A. M., Long, T. E., Showalter, T. N. & Libby, B. Thiol-Michael ‘click’ hydrogels as an malleable packing material for cancer therapy. Polymer. 125, 66-75, doi:10.1016/j.polymer.2017.07.078 (2017).
10. Pogoda, K., Chin, L., Georges, P. C., Byfield, F. J., Bucki, R., Kim, R., . . . Janmey, P. A. (2014). Compression stiffening of brain and its effect on mechanosensing by glioma cells. New J Phys, 16, 075002. doi:10.1088/1367-2630/16/7/075002.

### Example 3

Glioblastoma multiforme (GBM) is the most aggressive and lethal type of primary brain tumor in adults, accounting for 57.3% of all gliomas with an age-adjusted incident rate of 3.22 cases per 100,000 people [1]. The World Health Organizes categorizes GBM as a grade IV astrocytoma, and it is characterized by high infiltrative capacities and accelerated proliferation rates [2]. The standard treatment of care for patients upon diagnosis includes maximum surgical resection to remove the primary tumor mass, but since GBM tumors possess poorly defined borders and margins to result in incomplete resection [3], adjuvant chemotherapy with the oral DNA-alkylating drug temozolomide and radiotherapy [4] are administered to eradicate any remaining cancer cells and prevent tumor recurrence [5]. However, chemotherapeutic agents for treating GBM are limited to those that are able to cross the blood-brain-barrier [6]. Most chemotherapeutic drugs also have limited anti-tumor efficacy due to poor penetration into tumors and short retention times, and the heterogeneous populations of GBM cells enable the acquisition of chemoresistance [5, 7]. In addition, the dose of radiotherapy needed to treat the tumor surpasses the tolerance for healthy brain tissue and leads to necrosis [6]. Even with these combinatorial therapies, residual GBM cells remain to invade the brain parenchyma by traveling through white matter tracts and blood vessels, leading to the formation of new tumors within 2-3 cm of the resection cavity [8, 9]. Hence, tumor recurrences develop in 90%-95% of patients and result in dismal median survival times between 14-15 months [10, 11]. Furthermore, recent research has implicated that a GBM tumor cell subpopulation with stem-cell like properties is also responsible for recurrence [12]. These glioma cancer stem cells (GSCs) initiate tumor growth [13] and are resistant to therapy, with compelling data revealing GBM eradication requires their elimination [14]. GSC eradication is currently difficult due to a lack of established surface biomarkers for selective targeting [15]. Strategies to effectively eradicate both GSCs and GBM cells are therefore urgently needed to minimize tumor recurrence without damaging the brain microenvironment.

Most GBM research focuses on “search and destroy” techniques by treating the cancer cells as targets to be eliminated [16]. For example, Schiapparelli and colleagues have developed a self-assembling hydrogel that can release camptothecin prodrug over time after being directly applied to the tumor cavity post-surgery [17]. The researchers' in vivo work with mice demonstrated that drug release can be prolonged in the brain parenchyma to reduce tumor recurrence and improve survival. Although these types of localized treatments such as radioactive inflatable balloons, radioactive seeds, and biodegradable wafers loaded with chemotherapeutic agents have been tested on patients, randomized clinical studies did not improve outcomes [6]. Furthermore, these strategies do not effectively eradicate both the GBM and GSC populations of cancer cells. As such, there has been a shift in recent initiatives to focusing more on “attract to kill” strategies [16] by harnessing the infiltrative nature of GBM to guide cancer cell migration to scaffold implants for subsequent eradication.

Autier and colleagues developed a bacterial cellulose-based hydrogel scaffold for implantation in the tumor resection cavity to release chemokines and generate a chemical gradient to attract and immobilize GBM cells with surface adhesion [18]. However, the hydrogel is not degradable and brain slice invasion assays revealed the chemotactic gradient was diffusion limited and not high enough to induce cancer cell migration beyond 1-5 mm. In a separate study, Jain and colleagues engineered polycaprolactone nanofibers to mimic blood vessels and white matter tracts and guide GBM cells away from the primary tumor mass to an extracortical, cyclopamine drug-conjugated, collagen-based hydrogel to induce apoptosis in both GBM and GSCs, without toxicity to normal brain cells. And yet, this conduit is highly invasive and if compromised, could increase risk of enabling infiltration of the migrating tumor cells into healthy brain tissue to result in secondary tumor sites in the brain.

We are developing a novel and improved “attract to kill” technique to treat glioblastoma tumors. We propose that post-resection, an injectable hydrogel can be directed into the empty resection cavity after tumor removal. The biomaterial will be a viscous solution ex vivo and will undergo solution-gel transition inside the cavity. Unlike the aforementioned implants from other research groups, injectable hydrogels conform to patient anatomy to fill irregularly shaped spaces uniformly [6]. The hydrogel will be loaded with chemokines that both GSCs and GBM cells can sense, and the slow release of these chemoattractants will generate a chemotactic gradient to attract these residual, migratory cancer cells toward the scaffold through chemotaxis. A direct current, sub-lethal electric field generated by minimally invasive microelectrodes placed in the hydrogel and the surrounding brain tissue will further sensitize and preferentially direct the GBM and GSCs to migrate to the scaffold instead of other cells in the microenvironment, such as astrocytes. Focal adhesion of the cancer cells to the scaffold will allow the cells to pass through the hydrogel mesh to become entrapped inside. Upon localization and entrapment of the cancer cells in the hydrogel, focused ultrasound will be used to non-invasively ablate and eradicate the malignant cells. A schematic representation of this novel treatment method is outlined by FIG. 1.

This study is focused on optimizing and characterizing the injectable hydrogel platform for capturing and retaining the cancer cells during this proposed GBM treatment. Injectable hydrogels need to undergo fast gelation in a clinically relevant and surgically feasible timeframe under biocompatible conditions. Click chemistry reactions are very well suited for this type of application, since they reach completion rapidly under mild conditions when two reagents are mixed together to selectively result in high yields of the final product [19, 20]. Furthermore, click reactions are not impacted by the presence of water or oxygen due to their bio-orthogonal nature, enabling them to occur in vivo without any interference from biological processes [21]. The thiol-Michael addition is a specific type of click chemistry that can be conducted under physiological temperatures and biocompatible pH [22] and is ideal for generating hydrogels that can encapsulate protein and cells in situ [23, 24]. These reactions can undergo gelation within minutes, do not produce any free radicals detrimental to cell viabilities, and can functionalize the hydrogels with cell-adhesion promoting peptides [25, 26]. The thiol-acrylate reaction in particular is most commonly employed in developing biodegradable hydrogels, as the resulting thioether linkage can undergo hydrolytic degradation [27]. Although maleimides can also be utilized, unlike acrylates, some maleimides can be neurotoxic [28]. Bases are one of the most efficient thiol-Michael addition catalysts, as they minimize the propensity for side reactions [27]. Under an alkaline environment due to the base catalyst, the thiol group is deprotonated to produce a strong, nucleophilic thiolate anion and a conjugate acid [27]. The thiolate anion (Michael donor) then attacks and conjugates to the β-carbon of the electron deficient α,β-unsaturated carbonyl (Michael acceptor) in the acrylate to form an intermediate carbon-based anion, which is basic and obtains a hydrogel from the conjugate acid to form the final thioether product [27].

PEG monomers are the most commonly employed and dominant monomer for synthesizing hydrogels, and its biocompatibility has enabled it to be FDA approved for in vivo implantation in humans [29, 30]. Pritchard and colleagues developed a thiol-Michael addition injectable hydrogel based on a poly(ethylene glycol) diacrylate (PEGDA) backbone and showed its suitability to sustain the release of payloads [31]. In a follow up study, the researchers used phosphate buffered saline solution as the base catalyst and demonstrated that varying the buffer pH and strength within the physiologic range could be used to alter the hydrogel gelation kinetics [32]. While the basicity and nucleophilicity of the catalyst certainly affects the thiol-Michael addition reaction rate, it is also important to carefully select the base catalyst for clinical applications. Azabarsamy and Anseth note that buffering agents like trethanolamine or HEPES may be toxic in vivo. In contrast, sodium bicarbonate is a benign and biocompatible base that does not adversely affect the biological environment during the reaction and gelation process [33].

Moon and colleagues used sodium bicarbonate as the base catalyst to synthesize an injectable PEGDA-based hydrogel with the three-arm, trithiol, PEG-based crosslinker ethoxylated trimethylolpropane tri-3-mercaptopropionate (Thiocure ETTMP) with a thiol-Michael addition reaction for use as a packing material during brachytherapy for gynecological cancers [33]. The team systematically investigated the impact of the PEGDA molecular weight and the stoichiometric ratio between the PEGDA and Thiocure on the hydrogel properties and determined that these two parameters influenced the hydrogel properties only minimally. The instead, the preliminary findings indicated that the concentration of the base catalyst and initial hydration level may impact the hydrogel properties to a greater degree. However, the research focused on characterizing the hydrogel for its suitability as an acute implant, since most clinically relevant timeframes from brachytherapy are less than two hours, after which point the hydrogel will be extracted from the vaginal cavity. In a separate study, Khan and coworkers also utilized a similar hydrogel platform based on the thiol-Michael addition reaction between PEGDA and Thiocure, with an extraceullar, sodium bicarbonate-based buffer to assess the impact of the polymer concentration and molar ratio of thiol and acrylate on the hydrogel formulations. While the researchers determined that a slight excess of thiol to acrylate was optimal for the hydrogel gelation and that the weight percent of the monomers modulated hydrogel properties, the researchers focused on optimizing their platform as a three-dimensional (3D) cell culture model for mimicking the in vivo environment during in vitro cancer studies [26].

Since Peach and colleagues [34] were able to demonstrate the suitability of this PEGDA and Thiocure based hydrogel platform for its ability to conform to patient specific anatomical structures in cadavers, its minimum heat production upon gelation, and the fast reaction times, we selected this hydrogel platform as the base biomaterial for capturing the cancer cells during our proposed GBM treatment. As such, in this study, we systematically vary the initial hydration level and sodium bicarbonate concentration across three levels to characterize each of the nine resulting formulations based on its physical, biological, and chemical compatibility with the GBM microenvironment and its suitability as a chronic implant upon injection in vivo. This Example at least identifies and establishes the parameter-property relationships between the hydration level and basicity of the environment to the hydrogel properties and demonstrates an optimal hydrogel formulation for use in e.g., GBM treatment.

### Materials and Methods

### 2.1 Hydrogel Synthesis

Poly(ethylene glycol) diacrylate (PEGDA) with numbered average molecular weight of 575 g/mol (Sigma Aldrich) and Thiocure ETTMP 1300 333L (donated by Bruno Bock Thiochemicals) was prewarmed to room temperature. Stock solutions of sodium bicarbonate NaHCO₃(Fisher Chemical) were prepared at 0.1 M, 0.175 M, and 0.25 M in high performance liquid chromatography (HPLC) water (Fisher Scientific). Hydrogels were prepared with a 1:1 stoichiometric ratio of thiol:acrylate with 0.300 g of PEGDA and 0.389 g of thiocure. Each reagent was massed appropriated in tared syringes. A volume of 0.230 mL, 0.690 mL, and 2.05 mL was used for the 25 wt %, 50 wt %, and 75 wt % hydrogels, respectively. All the reagent masses and the volumes of NaHCO₃ were scaled appropriately when necessary for synthesizing hydrogels of different volumes. Hydrogels were crosslinked in 6 dram vials (Fisher Scientific) at 37° C. in a water bath. In order to optimize the best method for synthesizing the hydrogels, a total of 16 different permutations and combinations were assessed. Either the PEGDA or Thiocure were first mixed with the base by vortexing or swirling. The other reagent was next injected into the initial mixture, and the final mixture in the vial was either vortexed, swirled, not mixed mechanically, mixed with a syringe, or mixed with a stirring rod. The optimal synthesis method was determined by evaluating the gelation times through the inversion method and hydrogel homogeneity. Upon determining the optimal synthesis method, all nine formulations of hydrogels at the three various hydration levels and NaHCO₃ concentrations were synthesized to assess the hydrogel gelation times with the inversion method.

### 2.2 Swelling Studies

Swelling data was obtained for all nine formulations of hydrogels prepared according to the protocol outlined in 2.1. Any loose debris were cut out from the hydrogels to obtain uniform hydrogel surfaces. The gel mass (mg) was recorded for each hydrogel, after which point a single hydrogel was submerged in 50 mL of 1× phosphate buffered saline solution (PBS) (Gibco) pre-equilibrated to 37° C. in a jar. Three separate hydrogels were synthesized as replicates for each formulation. The PBS submerged hydrogel was maintained at 37° C. in a water bath. At 10 minute intervals for a total of 3 hours, the hydrogel was taken out of the PBS, patted dry with a Kimwipe, the wet mass was obtain (mw), and placed back in the PBS. The swelling ratio was taken as the percent increase in the hydrogel's mass with absorption of PBS according to Equation [1] as follows:

$$Percent mass increase = \frac{{m}_{d} - {m}_{w}}{{m}_{d}} \times 100 \qquad Eq . 1$$

Hydrogels were monitored daily to determine time point at which the equilibrium swelling was achieved as well as to obtain the equilibrium swelling ratios when the maximum swelling was achieved [35].

### 2.3 Rheology

Hydrogels for each formulation were synthesized to have thicknesses of 2 mm. The synthesized hydrogels were then cut to 10 mm×10 mm squares. Four separate replicates were prepared for each formulation and swelled for 12 hours in 10 mL of 1×PBS at 37° C. Each hydrogel was kept in the PBS and maintained in a hydrated environment to prevent drying until rheological analysis. The hydrogel was placed on a 25 mm plate in an RSA-G2 solids analyzer (TA Instruments) to obtain rheological data under the compression mode. A strain sweep from 1%-5% at 37° C. and 1 Hz was performed on the first replicate hydrogel for each formulation to identify the region where the storage modulus is linear. A frequency sweep from 0.1 to 10 Hz was performed at 2% strain and 37° C. for each of the remaining three replicates to collect the data on the storage and loss modulus. All final data are the reported at 1 Hz, where the moduli where in the linear viscoelastic region. The data for the storage modulus (G′), loss modulus (G″), and tan δ were obtained from TA Trios software.

### 2.4 Gel Fraction

Prepare 1 mL volume hydrogels by using a 12 well plate as the mold. Prepare 3 replicates for each formulation. After gelation, transfer the hydrogel to an individual watch glass (Fisher Scientific). Record the mass of the as formed hydrogel and air dry at room temperature for 24 hours. Record the mass, flip the hydrogel over to the other side, and dry in a vacuum desiccator for 24 hours at room temperature. Record the mass, flip the hydrogel to the other side, and dry in a vacuum desiccator for another 24 hours. If the difference in masses between the 24 hours and 48 hours of drying under vacuum are less than 0.01 g, consider the hydrogels dry and record the dry mass (mdry). If the difference is higher, dry under vacuum for another 24 hours and record the dry mass of the hydrogel afterwards. Transfer the hydrogel to a jar vial and submerge in 25 mL of dichloromethane (Millipore Sigma). Place the sample in an incubating mini shaker (Fisher Scientific) at room temperature at 150 rpm for 24 hours. Transfer hydrogel to a watch glass and air dry at room temperature for 24 h. Record mass of hydrogel and vacuum dry in a desiccator for 24 hours. Record mass of hydrogel and vacuum dry in a desiccator for another 24 hours. Hydrogels are considered dry if the difference in masses between the 24 hours and 48 hours of vacuum drying are less than 0.01 g. If dry, record final extracted hydrogel mass (mex). If not dry, dry under vacuum for another 24 hours before taking note of the extracted hydrogel mass. The percentage gel fraction for the hydrogel can be determined using the Equation [2] as follows:

$$Gel Fraction ( \% ) = \frac{{m}_{ex}}{{m}_{dry}} \times 100 \qquad Eq . 2$$

### 2.5 Disintegration

For each hydrogel formulation, 1 mL volumes were prepared in 9.5 dram vials. Three hydrogel replicates were prepared for each time point: days 0, 3, 6, 9, 12, and 15 for a total of 18 hydrogels per formulation. The masses of each as prepared hydrogel were recorded. Day 0 hydrogels were placed in a watch glass and air dried for 24 hours and then vacuum dried in a desiccator for 48 hours at room temperature. The hydrogels were dried further as needed until the change in mass was les than 0.01 g. The final dried mass for day 0 was recorded (m0). For the remaining hydrogels, 14 mL of 1×PBS (containing magnesium and calcium) pre-equilibrated to 37° C. at room temperature was added to the dram vial containing the hydrogel. The hydrogels in the vials were placed in the incubating mini shaker at 150 rpm and 37° C. At the designated time point, the replicates corresponding to the time point were taken out from the vials, washed three times with 10 mL of HPLC DI water to remove residual salt from the surface [31], and placed in a watch glass to dry. The hydrogels were air dried for 24 hours and then vacuum dried for 48 hours or more, until the change in mass was less than 0.01 g. The final dried mass for each time point was recorded (mf). For hydrogels that remained, the total volume of PBS was refreshed with 14 mL. The disintegration of the hydrogel at each time point was considered as a percentage mass loss with respect to the dried hydrogels at day 0 according to Equation [3] as follows:

$$Percent mass loss = \frac{{m}_{o} - {m}_{f}}{{m}_{o}} \times 100 \qquad Eq . 3$$

### 2.6 Cell Culture

Normal Human Astrocytes (NHA) from Lonza were cultured in Astrocyte Growth Medium (Lonza) at densities of 5000 cells/cm2 in flasks and maintained in incubator at 37° C. and 5% CO2. Media was replenished every other day according to manufacturer's protocol. Cells were subcultured upon reaching 80% confluence according to manufacturer's protocol. Briefly, cells were washed with HEPES buffer and incubated with Trypsin/EDTA for 4 min at 37° C. until 90% of cells were trypsinized from flask. The cell suspension was neutralized with Trypsin Neutralizing Solution and washed with HEPES buffer before centrifugation at 180 g for 5 minutes at 8° C. The cell pellet was resuspended in the Astrocyte Growth Medium (AGM) and a cell count with a hemocytometer was performed to quantify the cell density in the cell suspension. Cells from passages 5-7 were used for all experiments.

### 2.7 Sterile Hydrogel Synthesis

Sterile hydrogels were prepared inside a biosafety cabinet under aseptic conditions with auclaved sterile vials and UV radiation sterilized equipment. HPLC DI water was autoclaved before dissolving the appropriate mass of NaHCO₃. This base solution, PEGDA, and Thiocure solutions were all sterile filtered with polyethersulfone membrane 0.22 μm filter units (Millex). Hydrogels were prepared as described previously. Each hydrogel comprised a volume of 500 μL dispensed into a well in a 24 well plate and placed in an incubator to crosslink at physiologic temperature. Each hydrogel was submerged in 1 mL of AGM for 24 hours to pre-equilibrate hydrogels prior to use.

### 2.8 Cytotoxicity Assay

The alamarBlue (AB) assay (BioRad) was used to quantify cytotoxicity of the hydrogels to NHA by assessing the cell viability according to the manufacturer's protocol. The cell viabilities were determined by seeding NHA on the hydrogels and conducting the assay on days 1, 3, 5, and 7. Pre-equilibrated media in hydrogels were aspirated out and subcultured NHA were seeded on the hydrogel surface at a density of 50,000 cells per hydrogel. Each well was filled to 1.5 mL with fresh media. A positive control with 50,000 NHA in a well without any hydrogel was used to assess cell viabilities in a 2D environment. Four replicates of hydrogels were used for each time point separately for each formulation, for a total of 16 hydrogels in wells. Four replicates were also used for each time point separately for the 2D condition. The AB incubation step and quantification was performed for each of the 4 replicates in a formulation on the designated time point (day 1, 3, 5, and 7). Briefly, the media was refreshed for the wells of interest and AB was added at a volume of 10%. Corresponding 4 replicates of negative control with just media and AB without any cells were also prepared in 24 well plates for each time point. The incubation of AB with the samples and controls were conducted in the incubator for 4 hours. All samples were prepared and kept in the dark to minimize interference from light. For each replicate, 3 subsamples of the solution were obtained and dispensed in a 96 well plate. The spectrophotometer absorbance readings of all samples in the 96 well plate were determined at both 570 nm and 600 nm. AGM media was used as a blank to account for background absorbances. A standard curve with spectrophotometer data of NHA at various cell densities in 2D was used to convert the absorbance data to cell densities to quantify the cell viability of NHA over time, according to the manufacturer's protocol. Final cell viabilities reported were cell densities normalized to the cell seeding densities on the hydrogels.

### 2.9 IL-6 Secretion Immunoassay

In order to quantify the immunogenicity of NHA in response to the hydrogels, an ELISA was performed to measure IL-6 secretion. For each formulation, three sterile hydrogels were synthesized in 24 well plate wells according to the protocol outlined previously. Three additional sterile hydrogels were synthesized for each formulation to serve as negative controls. All hydrogels were incubated with 2 mL of AGM at 37° C. for 24 hours to allow pre-equilibration. 50,000 NHA were seeded on top of each hydrogel, while no cells were seeded on the negative controls. Three replicates of positive controls with the same seeding density of NHA on 24 well plate wells served as the positive control. All samples were maintained and cells cultured in an incubator at 37° C. with 5% CO. At designated time points (days 1, 3, 5, 7, 9, 11, 13, and 15), the entire supernatant from the samples was collected and centrifuged at 1500 rpm for 10 minutes at room temperature. 1.5 mL of the supernatant was collected, transferred to a fresh tube, and stored at −80° C. A scratch wound was performed on the positive control samples on Day 5, when the NHA reached full confluence in the well plate [36]. After collecting the supernatant for Day 5 positive control samples, 1 mL of PBS was added to each well and a sterile 200 μL pipette tip was dragged across the surface of the plate as a vertical line through the middle of the well. The PBS was removed rapidly and the wells were washed 2× with PBS to remove any cell debris before adding fresh media to the well. All samples were refreshed with 2 mL of AGM every other day for the entire duration of the experiment.

Human IL-6 DuoSet ELISA kit (R&D Systems) was used to quantify the IL-6 levels in each supernatant according to the manufacturer's protocol. Briefly, ELISA reagents and frozen supernatant samples were brought to room temperature prior to use. For each replicate, two subsamples were performed during the immunosassay. The 96 well plates were coated with mouse anti-human IL-6 capture antibody and incubated at room temperature overnight for 16 hours. After vigorously washing each well 3 times with wash buffer, the wells were blocked for 1 hour with 1% BSA (1× reagent diluent) solution at room temperature. All samples and the 7 point standards were subjected to a two-fold dilution in the reagent diluent. The wells were washed vigorously three times with wash buffer before being incubated with the samples and standards for two hours at room temperature. The wells were again washed three times with wash buffer and then incubated with biotinylated goat anti-human IL-6 detection antibody for 2 hours at room temperature. The samples were washed 3 times with wash buffer and then incubated with streptavidin-HRP for 20 minutes at room temperature in the dark. The samples were washed 3 times with wash buffer and incubated with a 1:1 mixture of chromogen and stabilized peroxide solution for 15 minutes before the reaction was stopped immediately with 2 N with sulfuric acid. Spectrophotometer microplate readings were taken at 540 nm and these absorbance values were subtracted from readings taken at 450 nm to account for wavelength corrections. Absorbance readings with reagent diluent served as the blank to account for any background absorbance signals in the assay. The 4P logistic sigmoid curve fit was used to model the standards and obtain the IL-6 secretions from the absorbance readings.

### 2.10 Immunocytochemistry

NHA were cultured on hydrogels and in 24 well plates, and the GFAP expression and cell morphology was monitored with confocal imaging to determine astrogliosis. The hydrogels were synthesized inside polydimethylsiloxane (PDMS) molds according to the protocol outlined by Ivey and colleagues [37]. Briefly, a mixture of 90% w/w Sylgard 184 Silicone Elastomer base with 10% w/w Sylgard 184 Silicone Elastomer curing agent was prepared and degassed in a vacuum desiccator for 30 minutes to remove any bubbles. The mixture was cured in a custom lithography printed metal plate at 100° C. for 30 minutes to yield PDMS stamps that were 10 mm in diameter and 1 mm in depth. Each stamp was autoclaved, placed inside a 24 well plate well and plasma treated for 4 minutes, and then sterilized by UV radiation for another 30 minutes to ensure sterility. The PDMS stamps were further treated by incubating with sterile 1% polyethylenimine for 10 minutes, followed by aspiration and incubation with sterile 0.1% glutaraldehyde for 20 min and washing with sterile DI water. Sterile hydrogels were synthesized according to the procedure described previously and 90 uL was dispensed into each PDMS stamp. The hydrogels were crosslinked at 37° C. for at least 30 minutes before each well was filled with 1 mL of AGM. The media was allowed to equilibrate with the hydrogels for 24 hours at 37° C. prior to cell seeding.

For the 2D condition, a custom 3D printed PLA, hollow well plate was built. Each well had a diameter of 14 mm and a depth of 12 mm. The bottom of each well was fitted with a single No. 1.5 thickness 18 mm×18 mm glass coverslip (Fisher Scientific) and the well plates were plasma treated for 4 minutes, followed by 70% ethanol treatment and then UV sterilization for 30 minutes in a biosafety cabinet. The coverslips were coated with 2% gelatin solution according to the manufacturer's protocol (Sigma Aldrich) to allow NHA to adhere to the coverslip and image the cells directly on the confocal microscope. For confocal imaging analysis, the samples were stained and imaged on Days 1, 3, 5, 7, 9, 11, 13, and 15 for a total of 8 time points. For each time point, three replicates of PDMS-hydrogel composites were synthesized for each formulation of the hydrogel and the 2D condition. A total of 20,000 NHA were seeded on the glass coverslip wells for the 2D condition, while 100,000 NHA were seeded on the PDMS-Hydrogel composites. For the 3D composites, the NHA were dispensed at the center to maximize the number of cells that will adhere to the hydrogel surface and minimize the number of cells that will adhere to the PDMS mold. All samples were maintained in 1 mL of AGM, which was refreshed every other day. At designated time points, the media for the samples corresponding to the time point were removed and the samples were washed three times with 1×PBS. The samples were fixed by incubating with 10% formalin for 20 minutes at room temperature, followed by another three washes and permeabilization with 0.5% TritonX-100 for 20 minutes. After another three washes, the samples were blocked by incubating with 1% BSA for 1 hour at room temperature, followed by another three washes. The samples were incubated with the GFAP monoclonal antibody GA5 (Life Technologies) at a 1:200 dilution in 1% BSA overnight in the dark at 4° C. The samples were then washed three times and incubated with goat anti-mouse IgG1 cross-adsorbed secondary Alexa Fluor 488 antibody at a 1:1000 dilution in 1% BSA for 1 hour at room temperature in the dark. The samples were washed three times and incubated with DAPI as a counterstain. Samples that were 3D (PDMS-hydrogel composites) were incubated with DAPI for 30 minutes, while 2D condition samples were incubated for 10 minutes at room temperature in the dark. The cells were again washed three times prior to imaging. Samples were imaged to under the 40×objective in a confocal microscope (Zeiss) to obtain data on the cell morphology and GFAP expression. Three fields of view were randomly selected per sample replicate for imaging. The normalized GFAP expression was quantified based on the ratio of the average fluorescence per pixel in each cell to the average fluorescence intensity in the background and average cell diameters were both obtained using the Zen Lite software.

### Results

### 3.1 Hydrogel Synthesis

### 3.2 Swelling Studies

Upon implantation of a hydrogel in brain tissue, cerebrospinal fluid can diffuse into the hydrogel and cause it to swell. The hydrogels were swelled in PBS to mimic and maintain physiologic pH and salinity. According to the results from the swelling kinetics (FIG. 1A), all the hydrogel formulations swelled over the course of 180 minutes, except for the three formulations with 75 wt % water content. The 75 wt % hydrogels deswelled and exhibited the highest variation across replicates. While both the 0.175 M and 0.25 M hydrogels at this hydration level reached a maximum swelling within the first 10 minutes, the 0.1 M hydrogel never reached maximum, defined as equilibrium swelling [35], due to continuous loss in mass over time (FIG. 1B). While the other six formulations of hydrogels were transparent under swelling conditions, the three 75 wt % hydrogels became cloudy, opaque, and adhesive under the same swelling conditions (FIG. 1C). The 25 wt % hydrogels swelled the most, with the formulation at 0.1 M NaHCO₃ swelling at the highest level. The 50 wt % hydrogel formulations all swelled the least and had the lowest variation across replicates. The 0.175 M hydrogel with 50 wt % water content took the longest time to reach equilibrium swelling (Table 1). Hydrogel mesh sizes based on rubber elastic theory are shown in Table 2.

TABLE 1
Gelation time and time to reach equilibrium
swelling for hydrogel formulations.

| Water Content | Base Concentration | Average Gelation | Time to Reach |
|---|---|---|---|
| (%) | (M) | Time (s) | Equilibrium |
| 25 | 0.1 | 856 ± 19.8 | 77.5 |
| | 0.175 | 45.5 ± 0.7 | 2.5 |
| | 0.25 | 21 ± 1.4 | 3 |
| 50 | 0.1 | 37.5 ± 1 | 3 |
| | 0.175 | 41 ± 2.5 | 102.5 |
| | 0.25 | 14 ± 1.2 | 76 |
| 75 | 0.1 | 35.5 ± 0.6 | 0 |
| | 0.175 | 41 ± 1.1 | 0.167 |
| | 0.25 | 36.5 ± 1.5 | 0.167 |

TABLE 2
Hydrogel mesh sizes based on rubber elastic theory

| Water Content | Base Concentration | Average Mesh |
|---|---|---|
| (%) | (M) | Size (nm) |
| 25 | 0.1 | 8.63 ± 2.43 |
| | 0.175 | 8.21 ± 2.80 |
| | 0.25 | 8.80 ± 1.29 |
| 50 | 0.1 | 10.95 ± 2.28 |
| | 0.175 | 5.60 ± 0.58 |
| | 0.25 | 4.16 ± 0.84 |

### Discussion

Characterizing the hydrogel swelling can provide many important information for biomedical applications, since these materials thermodynamically interact with aqueous media in a manner similar to the ECM [31]. Excessive hydrogel swelling can damage adjacent neurons [38], be detrimental to patient safety [39], and decrease the performance of the hydrogel in vivo [31]. The results from the swelling studies overall indicated that the initial hydration level of the hydrogels had a greater impact on its swelling characteristics than the initial concentration of NaHCO₃, as a decrease in the initial hydration level led to an increase in swelling, as previously observed by Moon and colleagues [33]. All three formulations of the 75 wt % hydrogels possessed a water content above the equilibrium swelling ratio, which is why these formulations had a tendency to deswell during the course of the swelling kinetics study. Swelling data can determine hydrogels with batch-to-batch variations [40], and these three formulations possessed the greatest variation in data, as evidenced by the large error bars in FIG. 1A. These findings, taken together with the change in opacity of the 75 wt % hydrogels in PBS, indicate that these formulations were poorly crosslinked. The increased water content increased the volume of sodium bicarbonate during the synthesis process, lowered the hydrogel cloud point and led to a separation of phases. Pritchard and colleagues had similarly observed poor polymerization in hydrogel formulations with increased salinity, due to a lower cloud point in their hydrogels [31]. Since the hydrogels need to be stable as chronic implants, the 75 wt % hydrogels were screened out from further characterizations. In contrast, the hydrogels at 50 wt % exhibited the least batch-to-batch variability and swelling. Since hydrogel swelling is inversely proportional to its crosslinking density [41], the 50 wt % hydrogels had the highest crosslinking density. The 25 wt % hydrogel formulations swelled the most, since the initial hydration level was the furthest from the equilibrium swelling point. Specifically, the 0.1 M hydrogel at 25 wt % swelled the most and had the lowest crosslinking density. This result is not surprising, considering this hydrogel also takes the longest time to undergo gelation. The NaHCO₃ base catalyst content during synthesis is the lowest for this formulation due to the low water content and base concentration, which is why this formulation takes too long to crosslink and possesses a weaker crosslinking density. The 0.175 M hydrogel at 50 wt % takes the longest time to reach the swelling equilibrium point, which can be beneficial for the surrounding tissue and enable time for cells to gradually adjust to the expanding hydrogel.

### References for Example 3

[1] Q. T. Ostrom, G. Cioffi, H. Gittleman, N. Patil, K. Waite, C. Kruchko, J. S. Barnholtz-Sloan, CBTRUS Statistical Report: Primary Brain and Other Central Nervous System Tumors Diagnosed in the United States in 2012-2016, Neuro-oncology 21(Suppl 5) (2019) v1-v100.
[2] E. C. Holland, Glioblastoma multiforme: the terminator, Proceedings of the National Academy of Sciences of the United States of America 97(12) (2000) 6242-4.
[3] T. Ozeki, D. Kaneko, K. Hashizawa, Y. Imai, T. Tagami, H. Okada, Improvement of survival in C6 rat glioma model by a sustained drug release from localized PLGA microspheres in a thermoreversible hydrogel, Int J Pharm 427(2) (2012) 299-304.
[4] A. Bagherian, R. Mardani, B. Roudi, M. Taghizadeh, H. R. Banfshe, A. Ghaderi, A. Davoodvandi, S. Shamollaghamsari, M. R. Hamblin, H. Mirzaei, Combination Therapy with Nanomicellar-Curcumin and Temozolomide for In Vitro Therapy of Glioblastoma Multiforme via Wnt Signaling Pathways, Journal of Molecular Neuroscience 70(10) (2020) 1471-1483.
[5] H. L. Xu, K. L. Mao, C. T. Lu, Z. L. Fan, J. J. Yang, J. Xu, P. P. Chen, D. L. ZhuGe, B. X. Shen, B. H. Jin, J. Xiao, Y. Z. Zhao, An injectable acellular matrix scaffold with absorbable permeable nanoparticles improves the therapeutic effects of docetaxel on glioblastoma, Biomaterials 107 (2016) 44-60.
[6] P. Puente, N. Fettig, M. J. Luderer, A. Jin, S. Shah, B. Muz, V. Kapoor, S. M. Goddu, N. N. Salama, C. Tsien, D. Thotala, K. Shoghi, B. Rogers, A. K. Azab, Injectable Hydrogels for Localized Chemotherapy and Radiotherapy in Brain Tumors, J Pharm Sci 107(3) (2018) 922-933.
[7] C. Bastiancich, J. Bianco, K. Vanvarenberg, B. Ucakar, N. Joudiou, B. Gallez, G. Bastiat, F. Lagarce, V. Preat, F. Danhier, Injectable nanomedicine hydrogel for local chemotherapy of glioblastoma after surgical resection, J Control Release 264 (2017) 45-54.
[8] M. Lara-Velazquez, R. Al-Kharboosh, S. Jeanneret, C. Vazquez-Ramos, D. Mahato, D. Tavanaiepour, G. Rahmathulla, A. Quinones-Hinojosa, Advances in Brain Tumor Surgery for Glioblastoma in Adults, Brain Sci 7(12) (2017).
[9] L. E. Gaspar, B. J. Fisher, D. R. Macdonald, D. V. LeBer, E. C. Halperin, S. C. Schold, Jr., J. G. Cairncross, Supratentorial malignant glioma: patterns of recurrence and implications for external beam local treatment, Int J Radiat Oncol Biol Phys 24(1) (1992) 55-7.
[10] P. De Bonis, C. Anile, A. Pompucci, A. Fiorentino, M. Balducci, S. Chiesa, L. Lauriola, G. Maira, A. Mangiola, The influence of surgery on recurrence pattern of glioblastoma, Clinical Neurology and Neurosurgery 115(1) (2013) 37-43.
[11] M. R. Gilbert, M. Wang, K. D. Aldape, R. Stupp, M. E. Hegi, K. A. Jaeckle, T. S. Armstrong, J. S. Wefel, M. Won, D. T. Blumenthal, A. Mahajan, C. J. Schultz, S. Erridge, B. Baumert, K. I. Hopkins, T. Tzuk-Shina, P. D. Brown, A. Chakravarti, W. J. Curran, Jr., M. P. Mehta, Dose-dense temozolomide for newly diagnosed glioblastoma: a randomized phase III clinical trial, J Clin Oncol 31(32) (2013) 4085-91.
[12] D. Garnier, O. Renoult, M. C. Alves-Guerra, F. Paris, C. Pecqueur, Glioblastoma Stem-Like Cells, Metabolic Strategy to Kill a Challenging Target, Front Oncol 9 (2019) 118.
[13] S. K. Singh, C. Hawkins, I. D. Clarke, J. A. Squire, J. Bayani, T. Hide, R. M. Henkelman, M. D. Cusimano, P. B. Dirks, Identification of human brain tumour initiating cells, Nature 432(7015) (2004) 396-401.
[14] T. Sun, H. Wu, Y. Li, Y. Huang, L. Yao, X. Chen, X. Han, Y. Zhou, Z. Du, Targeting transferrin receptor delivery of temozolomide for a potential glioma stem cell-mediated therapy, Oncotarget 8(43) (2017) 74451-74465.
[15] T. Glaser, I. Han, L. Wu, X. Zeng, Targeted Nanotechnology in Glioblastoma Multiforme, Front Pharmacol 8 (2017) 166.
[16] B. van der Sanden, F. Appaix, F. Berger, L. Selek, J. P. Issartel, D. Wion, Translation of the ecological trap concept to glioma therapy: the cancer cell trap concept, Future Oncol 9(6) (2013) 817-24.
[17] P. Schiapparelli, P. Zhang, M. Lara-Velazquez, H. Guerrero-Cazares, R. Lin, H. Su, R. W. Chakroun, M. Tusa, A. Quiñones-Hinojosa, H. Cui, Self-assembling and self-formulating prodrug hydrogelator extends survival in a glioblastoma resection and recurrence model, Journal of Controlled Release 319 (2020) 311-321.
[18] L. Autier, A. Clavreul, M. L. Cacicedo, F. Franconi, L. Sindji, A. Rousseau, R. Perrot, C. N. Montero-Menei, G. R. Castro, P. Menei, A new glioblastoma cell trap for implantation after surgical resection, Acta Biomater 84 (2019) 268-279.
[19] Y. Zou, L. Zhang, L. Yang, F. Zhu, M. Ding, F. Lin, Z. Wang, Y. Li, “Click” chemistry in polymeric scaffolds: Bioactive materials for tissue engineering, Journal of Controlled Release 273 (2018) 160-179.
[20] Z. Xu, K. M. Bratlie, Click Chemistry and Material Selection for in Situ Fabrication of Hydrogels in Tissue Engineering Applications, ACS Biomaterials Science & Engineering 4(7) (2018) 2276-2291.
[21] C. Echalier, L. Valot, J. Martinez, A. Mehdi, G. Subra, Chemical cross-linking methods for cell encapsulation in hydrogels, Materials Today Communications 20 (2019) 100536.
[22] K. R. Coogan, P. T. Stone, N. D. Sempertegui, S. S. Rao, Fabrication of micro-porous hyaluronic acid hydrogels through salt leaching, European Polymer Journal 135 (2020).
[23] K. Shah, D. Vasileva, A. Karadaghy, S. P. Zustiak, Development and characterization of polyethylene glycol-carbon nanotube hydrogel composite, J Mater Chem B 3(40) (2015) 7950-7962.
[24] M. P. Lutolf, G. P. Raeber, A. H. Zisch, N. Tirelli, J. A. Hubbell, Cell-Responsive Synthetic Hydrogels, Advanced Materials 15(11) (2003) 888-892.
[25] E. A. Phelps, K. L. Templeman, P. M. Thule, A. J. Garcia, Engineered VEGF-releasing PEG-MAL hydrogel for pancreatic islet vascularization, Drug Deliv Transl Res 5(2) (2015) 125-36.
[26] A. H. Khan, J. K. Cook, W. J. Wortmann, 3rd, N. D. Kersker, A. Rao, J. A. Pojman, A. T. Melvin, Synthesis and characterization of thiol-acrylate hydrogels using a base-catalyzed Michael addition for 3D cell culture applications, J Biomed Mater Res B Appl Biomater 108(5) (2020) 2294-2307.
[27] D. P. Nair, M. Podgórski, S. Chatani, T. Gong, W. Xi, C. R. Fenoli, C. N. Bowman, The Thiol-Michael Addition Click Reaction: A Powerful and Widely Used Tool in Materials Chemistry, Chemistry of Materials 26(1) (2013) 724-744.
[28] A. B. Lowe, Thiol-ene “click” reactions and recent applications in polymer and materials synthesis, Polymer Chemistry 1(1) (2010) 17-36.
[29] C. C. Lin, K. S. Anseth, PEG hydrogels for the controlled release of biomolecules in regenerative medicine, Pharm Res 26(3) (2009) 631-43.
[30] R. G. Chapman, E. Ostuni, M. N. Liang, G. Meluleni, E. Kim, L. Yan, G. Pier, H. S. Warren, G. M. Whitesides, Polymeric Thin Films That Resist the Adsorption of Proteins and the Adhesion of Bacteria, Langmuir 17(4) (2001) 1225-1233.
[31] C. D. Pritchard, T. M. O'Shea, D. J. Siegwart, E. Calo, D. G. Anderson, F. M. Reynolds, J. A. Thomas, J. R. Slotkin, E. J. Woodard, R. Langer, An injectable thiol-acrylate poly(ethylene glycol) hydrogel for sustained release of methylprednisolone sodium succinate, Biomaterials 32(2) (2011) 587-97.
[32] T. M. O'Shea, A. A. Aimetti, E. Kim, V. Yesilyurt, R. Langer, Synthesis and characterization of a library of in-situ curing, nonswelling ethoxylated polyol thiol-ene hydrogels for tailorable macromolecule delivery, Adv Mater 27(1) (2015) 65-72.
[33] N. G. Moon, A. M. Pekkanen, T. E. Long, T. N. Showalter, B. Libby, Thiol-Michael ‘click’ hydrogels as an imagable packing material for cancer therapy, Polymer 125 (2017) 66-75.
[34] M. S. Peach, J. Moore, W. Giles, J. Trainor, T. Long, N. Moon, J. E. Hylton, T. N. Showalter, B. Libby, Development and preclinical testing of a novel biodegradable hydrogel vaginal packing technology for gynecologic high-dose-rate brachytherapy, J Contemp Brachytherapy 10(4) (2018) 306-314.
[35] J. Sievers, K. Sperlich, T. Stahnke, C. Kreiner, T. Eickner, H. Martin, R. F. Guthoff, M. Schunemann, S. Bohn, O. Stachs, Determination of hydrogel swelling factors by two established and a novel non-contact continuous method, Journal of Applied Polymer Science 138(18) (2021) 50326.
[36] N. Wang, F. Yao, K. Li, L. Zhang, G. Yin, M. Du, B. Wu, Fisetin regulates astrocyte migration and proliferation in vitro, Int J Mol Med 39(4) (2017) 783-790.
[37] J. W. Ivey, E. L. Latouche, M. B. Sano, J. H. Rossmeisl, R. V. Davalos, S. S. Verbridge, Targeted cellular ablation based on the morphology of malignant cells, Scientific Reports 5(1) (2015) 17157.
[38] S. L. Blackburn, M. D. Smyth, Hydrogel-induced cervicomedullary compression after posterior fossa decompression for Chiari malformation. Case report, J Neurosurg 106(4 Suppl) (2007) 302-4.
[39] B. J. Neuman, K. Radcliff, J. Rihn, Cauda equina syndrome after a TLIF resulting from postoperative expansion of a hydrogel dural sealant, Clin Orthop Relat Res 470(6) (2012) 1640-1645.
[40] S. R. Caliari, J. A. Burdick, A practical guide to hydrogels for cell culture, Nature Methods 13(5) (2016) 405-414.
[41] Y. Liang, M. V. Coffin, S. D. Manceva, J. A. Chichester, R. M. Jones, K. L. Kiick, Controlled release of an anthrax toxin-neutralizing antibody from hydrolytically degradable polyethylene glycol hydrogels, J Biomed Mater Res A 104(1) (2016) 113-23.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An injectable hydrogel comprising:
a hydrogel matrix comprising one or more polymers;
a wt % of water that is any non-zero wt % that ranges from about 0 up to, but not including, 75 wt %;
optionally, one or more agents, wherein one of the one or more agents is optionally a cell migration modulator,
wherein the storage modulus of the injectable hydrogel ranges from about 3 to about 100 kPa, from about 3 to about 50 kPa, or from about 3 to about 25 kPa.
   2. The injectable hydrogel of aspect 1, wherein the wt % of water is any non-zero wt % ranging from about 0 to about 50 wt %, 0 to about 25 wt %, about 25 wt % to about 50 wt %, about 25 wt % up to, but not including 75 wt %, or about 50 wt % up to, but not including, 75 wt %.
   3. The injectable hydrogel of any one of aspects 1-2, wherein the injectable hydrogel has a storage modulus effective for implantation into brain tissue.
   4. The injectable hydrogel of any one of aspects 1-3, wherein the injectable hydrogel is biocompatible.
   5. The injectable hydrogel of any one of aspects 1-4, wherein the injectable hydrogel is responsive to a stimulus.
   6. The injectable hydrogel of any one of aspects 1-5, wherein the stimulus is an abiotic environmental condition, a chemical, a biologic agent, an energy, or any combination thereof.
   7. The injectable hydrogel of any one of aspects 1-6, wherein the injectable hydrogel is an agent eluting hydrogel and is capable of releasing one or more agents into the environment surrounding the hydrogel.
   8. The injectable hydrogel of any one of aspects 1-7, wherein the stimulus is capable of triggering agent elution from the hydrogel, agent activation, agent deactivation, or any combination thereof.
   9. The injectable hydrogel of any one of aspects 1-8, wherein the cell migration modulator is a cell attractant.
   10. The injectable hydrogel of aspect 9, wherein the cell attractant is
a. a cancer stem cell attractant;
b. a circulating cancer cell attractant;
c. a migrating cancer cell attractant;
d. a disseminating cancer cell attractant;
e. a glioma cell attractant;
f. a tumor microenvironment cell attractant;
g. an immune cell attractant;
h. a cancer cell attractant;
i. a cancer-associated fibroblast attractant;
j. a tumor initiating cell attractant; or
k. any combination thereof.
   11. The injectable hydrogel of any one of aspects 1-10, wherein the one or more polymers comprises a polymer having one or more hydrophilic groups.
   12. The injectable hydrogel of any one of aspects 1-11, wherein each of the one or more hydrophilic groups individually selected from the group consisting of: —NH₂, —COOH, —OH, —CONH₂, —CONH—, and —SO₃H.
   13. The injectable hydrogel of any one of aspects 1-12, wherein the injectable hydrogel is cationic, nonionic, or anionic.
   14. The injectable hydrogel of any one of aspects 1-13, wherein the one or more polymers comprises a natural polymer, a synthetic polymer, or a combination thereof.
   15. The injectable hydrogel of any one of aspects 1-14, wherein the one or more polymers are chemically crosslinked, physically crosslinked, or both.
   16. The injectable hydrogel of any one of aspects 1-15, wherein the one or more polymers are each individually selected from polyethylene glycol (PEG), chitosan, Poly(2-hydroxyethyl methacrylate) (PHEMA), 2-Hydroxyethyl methacrylate (HEMA), Hydroxyethoxyethyl metha-crylate (HEEMA), Hydroxydiethoxyethylmethacrylate (HDEEMA), Methoxyethyl methacrylate (MEMA), Methoxyethoxyethyl methacrylate (MEEMA), Methoxy-diethoxyethyl methacrylate (MDEEMA), Ethylene glycol dimethacrylate (EGDMA), N-vinyl-2-pyrrolidone (NVP), N-isopropyl AAm (NIPAAm), Vinyl acetate (VAc), Acrylic acid (AA), N-(2-hydroxypropyl) methacrylamide (HPMA), Ethylene glycol (EG), PEG acrylate (PEGA), PEG methacrylate (PEGMA), PEG diacrylate (PEGDA), PEG dimethacrylate (PEGDMA), Methacrylic acid (MAA), PEG-PEGMA, Carboxymethyl cellulose (CMC), Polyvinylpyrrolidone (PVP), an Acrylamide/acrylic acid copolymer, linear cationic polyallylammonium chloride, Poly(N-isopropyl acrylamide) (PNIPAM), self-assembling peptides, acrylate-modified PEG and acrylate-modified hyaluronic acid, heparin, amine end-functionalized 4-arm star-PEG, or any combination thereof.
   17. The injectable hydrogel of any one of aspects 1-16, wherein at least one of the one or more polymers is PEGDA.
   18. The injectable hydrogel of any one of aspects 1-17, wherein the injectable hydrogel is a thiol-Michael addition hydrogel.
   19. The injectable hydrogel of any one of aspects 1-18, wherein the injectable hydrogel is a reaction product of a polymer comprising at least one Michael acceptor and a thiol compound reacted in the presence of an aqueous base.
   20. The injectable hydrogel of any one of aspects 1-19, wherein the one or more polymers comprises a monomer that is a Michael acceptor.
   21. The injectable hydrogel of any one of aspects 19-20, wherein the Michael acceptor is acrylate, vinyl nitrile, vinyl nitro, vinyl phosphonate, vinyl sulfonate, or a compound comprising an enone.
   22. The injectable hydrogel of any one of aspects 19-21, wherein the thiol compound is a multi-arm, thiol terminated polymer comprising a backbone consisting of: poly(ethylene glycol), polycaprolactam, poly(propylene glycol), and poly(lactide) chains, and any water-soluble polysaccharide functionalized with 3 or more thiol groups per chain.
   23. The injectable hydrogel of any one of aspects 19-22, wherein the thiol compound is a multi-arm, thiol-terminated polyethylene glycol (PEG) oligomer or ethoxylated trimethylolpropane tri-3-mercaptopropionate.
   24. The injectable hydrogel of aspect 23, wherein the thiol-terminated PEG oligomer has an average molecular weight less than about 100,000 g/mol.
   25. The injectable hydrogel of any one of aspects 19-24, wherein the aqueous base is an inorganic carbonate, an inorganic bicarbonate, a buffer having a pH ranging from 7.4-14, an amine base, or any combination thereof.
   26. The injectable hydrogel of any one of aspects 19-25, wherein the aqueous base is NaHCO₃.
   27. The injectable hydrogel of any one of aspects 19-26, wherein the concentration of the aqueous base is 0.1 M-0.25 M.
   28. The injectable hydrogel of any one of aspects 1-27, wherein the injectable hydrogel is capable of capturing and/or retaining one or more cells.
   29. The injectable hydrogel of any one of aspects 1-28, wherein one or more of the one or more agents is selected from the group consisting of: DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, agent sensitizers, imaging agents, chemotherapeutic agents, chemokines, anti-migratory compounds capable of inhibit inhibiting chemokine receptors to decrease cell invasion, and any combination thereof.
   30. A method of attracting cells to and capturing cells an injectable hydrogel of any one of aspects 1-29 that is within a subject, the method comprising:
optionally releasing the optional cell migratory modulating agent from the injectable hydrogel so as to form a chemotaxis gradient in the environment around the hydrogel; and
applying a first external stimulus to the injectable hydrogel and/or body cavity of the subject, wherein the first external stimulus effective to stimulate migration of one or more cells within the subject to the injected injectable hydrogel; and capturing and/or retaining one or more of the one or more cells within the hydrogel for a period of time.
   31. The method of aspect 30, wherein the one or more cells are selected from: cancer cells, cancer stem cells, circulating cancer cells, residual cancer cells, tumor microenvironment cells, immune cells, tumor initiating cells, cancer-associated fibroblasts, or any combination thereof.
   32. The method of any one of aspects 30-31, wherein the method further comprises injecting, into a body cavity of a subject,
a. an injectable hydrogel of any of aspects 1-29, or
b. one or more reagents capable of forming an injectable hydrogel of any of aspects 1-29 so as to form an injected injectable hydrogel within the body cavity after injection.
   33. The method of any one of aspects 30-32, further comprising exposing the injected injectable hydrogel to a second external stimulus to the hydrogel after a period of time sufficient to capture and/or retain one or more cells in the injectable hydrogel.
   34. The method of aspect 33, wherein the second external stimulus is capable of modifying, modulating, inhibiting growth of, and/or killing one or more cells captured and/or retained in the injectable hydrogel.
   35. The method of any one of aspects 33-34, wherein the second external stimulus is an energy and wherein the energy is an electric energy, a light energy, a magnetic energy, a thermal energy, an acoustic energy, a chemical energy, a biochemical energy, a radiation energy, or any combination thereof.
   36. The method of any one of aspects 33-35, wherein the external stimulus is an acoustic energy or an electric energy.
   37. The method of aspect 36, wherein the acoustic energy is ultrasound.
   38. The method of aspect 36, wherein the electric energy is delivered by one or more probes, wherein
c. a cathode probe or cathode end of a probe is positioned in, adjacent to, or in proximity to the injectable hydrogel; or
d. an anode probe or anode end of a probe is positioned in, adjacent to, or in proximity to the injectable hydrogel.
   39. The method of aspect 38, wherein the one or more probes or other energy sources capable of delivering the second external stimulus are placed in operable proximity to the injected injectable hydrogel.
   40. The method of any one of aspects 30-39, wherein one or more of the one or more cells originates from the body cavity microenvironment.
   41. The method of any one of aspects 33-40, wherein the second external stimulus is capable of
a. damaging one or more cells attracted to, in immediate proximity of, contained in the injected injectable hydrogel, or any combination thereof;
b. killing or ablating one or more cells attracted to, in immediate proximity of, contained in the injected injectable hydrogel; or any combination thereof;
c. modifying the one or more cells attracted to, in immediate proximity of, contained in the injected injectable hydrogel, or any combination thereof, or
d. any combination thereof.
   42. The method of any one of aspects 30-41, wherein the body cavity is a surgical cavity.
   43. The method of aspect 42, wherein the surgical cavity is formed from removing a tumor from the body of the subject.
   44. The method of any one of aspects 42-43, wherein the body cavity is in the brain of the subject.
   45. The method of aspect 44, wherein the tumor is a glioma.
   46. A method of treating a cancer in a subject, comprising performing the method as in any one of aspects 30-45.
   47. The method of aspect 46, wherein the cancer is a glioblastoma.
   48. The method of aspect 47, wherein the glioblastoma is glioblastoma multiforme.
   49. The method of any one of aspects 46-48, further comprising imaging the injected injectable hydrogel after injecting into a subject.
   50. The method of any one of aspects 46-49, further comprising releasing one or more agents optionally included in the injectable hydrogel over a period of time.
   51. The method of any one of aspects 46-50, further comprising removing the injected hydrogel after one or more cells are collected therein.
   52. The method of any one of aspects 46-51, further comprising delivering to the subject one or more chemotherapeutics, therapeutic radiation, or both.

What is claimed is:

1. An injectable hydrogel comprising:
a hydrogel matrix comprising one or more polymers;
a wt % of water that is any non-zero wt % that ranges from about 0 up to, but not including, 75 wt %;
optionally, one or more agents, wherein one of the one or more agents is optionally a cell migration modulator,
wherein the storage modulus of the injectable hydrogel ranges from about 3 to about 100 kPa, from about 3 to about 50 kPa, or from about 3 to about 25 kPa.

2. The injectable hydrogel of claim 1, wherein the wt % of water is any non-zero wt % ranging from about 0 to about 50 wt %, 0 to about 25 wt %, about 25 wt % to about 50 wt %, about 25 wt % up to, but not including 75 wt %, or about 50 wt % up to, but not including, 75 wt %.

3. The injectable hydrogel of claim 1, wherein the injectable hydrogel has a storage modulus effective for implantation into brain tissue.

4. The injectable hydrogel of claim 1, wherein the injectable hydrogel is biocompatible.

5. The injectable hydrogel of claim 1, wherein the injectable hydrogel is responsive to a stimulus.

6. The injectable hydrogel of claim 5, wherein the stimulus is an abiotic environmental condition, a chemical, a biologic agent, an energy, or any combination thereof.

7. The injectable hydrogel of claim 6, wherein the injectable hydrogel is an agent eluting hydrogel and is capable of releasing one or more agents into the environment surrounding the hydrogel.

8. The injectable hydrogel of claim 7, wherein the stimulus is capable of triggering agent elution from the hydrogel, agent activation, agent deactivation, or any combination thereof.

9. The injectable hydrogel of claim 1, wherein the cell migration modulator is a cell attractant.

10. The injectable hydrogel of claim 9, wherein the cell attractant is
a. a cancer stem cell attractant;
b. a circulating cancer cell attractant;
c. a migrating cancer cell attractant;
d. a disseminating cancer cell attractant;
e. a glioma cell attractant;
f. a tumor microenvironment cell attractant;
g. an immune cell attractant;
h. a cancer cell attractant;
i. a cancer-associated fibroblast attractant;
j. a tumor initiating cell attractant; or
k. any combination thereof.

11. The injectable hydrogel of claim 1, wherein the one or more polymers comprises a polymer having one or more hydrophilic groups.

12. The injectable hydrogel of claim 11, wherein each of the one or more hydrophilic groups individually selected from the group consisting of: —NH₂, —COOH, —OH, —CONH₂, —CONH—, and —SO₃H.

13. The injectable hydrogel of claim 1, wherein the injectable hydrogel is cationic, nonionic, or anionic.

14. The injectable hydrogel of claim 1, wherein the one or more polymers comprises a natural polymer, a synthetic polymer, or a combination thereof.

15. The injectable hydrogel of claim 1, wherein the one or more polymers are chemically crosslinked, physically crosslinked, or both.

16. The injectable hydrogel of claim 1, wherein the one or more polymers are each individually selected from polyethylene glycol (PEG), chitosan, Poly(-hydroxyethyl methacrylate) (PHEMA), 2-Hydroxyethyl methacrylate (HEMA), Hydroxyethoxyethyl methacrylate (HEEMA), Hydroxydiethoxyethylmethacrylate (HDEEMA), Methoxy ethyl methacrylate (MEMA), Methoxyethoxyethyl methacrylate (MEEMA), Methoxy-diethoxyethyl methacrylate (MDEEMA), Ethylene glycol dimethacrylate (EGDMA), N-vinyl-2-pyrrolidone (NVP), N-isopropyl AAm (NIPAAm), Vinyl acetate (VAc), Acrylic acid (AA), N-(2-hydroxypropyl) methacrylamide (HPMA), Ethylene glycol (EG), PEG acrylate (PEGA), PEG methacrylate (PEGMA), PEG diacrylate (PEGDA), PEG dimethacrylate (PEGDMA), Methacrylic acid (MAA), PEG-PEGMA, Carboxymethyl cellulose (CMC), Polyvinylpyrrolidone (PVP), an Acrylamide/acrylic acid copolymer, linear cationic polyallylammonium chloride, Poly(N-isopropyl acrylamide) (PNIPAM), self-assembling peptides, acrylate-modified PEG and acrylate-modified hyaluronic acid, heparin, amine end-functionalized 4-arm star-PEG, or any combination thereof.

17. The injectable hydrogel of claim 16, wherein at least one of the one or more polymers is PEGDA.

18. The injectable hydrogel of claim 1, wherein the injectable hydrogel is a thiol-Michael addition hydrogel.

19. The injectable hydrogel of claim 18, wherein the injectable hydrogel is a reaction product of a polymer comprising at least one Michael acceptor and a thiol compound reacted in the presence of an aqueous base.

20. The injectable hydrogel of claim 19, wherein the one or more polymers comprises a monomer that is a Michael acceptor.

21. The injectable hydrogel of claim 20, wherein the Michael acceptor is acrylate, vinyl nitrile, vinyl nitro, vinyl phosphonate, vinyl sulfonate, or a compound comprising an enone.

22. The injectable hydrogel of claim 19, wherein the thiol compound is a multi-arm, thiol terminated polymer comprising a backbone consisting of: poly(ethylene glycol), polycaprolactam, poly(propylene glycol), and poly(lactide) chains, and any water-soluble polysaccharide functionalized with 3 or more thiol groups per chain.

23. The injectable hydrogel of claim 19, wherein the thiol compound is a multi-arm, thiol-terminated polyethylene glycol (PEG) oligomer or ethoxylated trimethylolpropane tri-3-mercaptopropionate.

24. The injectable hydrogel of claim 23, wherein the thiol-terminated PEG oligomer has an average molecular weight less than about 100,000 g/mol.

25. The injectable hydrogel of claim 19, wherein the aqueous base is an inorganic carbonate, an inorganic bicarbonate, a buffer having a pH ranging from 7.4-14, an amine base, or any combination thereof.

26. The injectable hydrogel of claim 25, wherein the aqueous base is NaHCO₃.

27. The injectable hydrogel of claim 26, wherein the concentration of the aqueous base is 0.1 M-0.25 M.

28. The injectable hydrogel of claim 1, wherein the injectable hydrogel is capable of capturing and/or retaining one or more cells.

29. The injectable hydrogel of claim 1, wherein one or more of the one or more agents is selected from the group consisting of: DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, agent sensitizers, imaging agents, chemotherapeutic agents, chemokines, anti-migratory compounds capable of inhibit inhibiting chemokine receptors to decrease cell invasion, and any combination thereof.

30. A method of attracting cells to and capturing cells an injectable hydrogel of any one of claims 1-29 that is within a subject, the method comprising:
optionally releasing the optional cell migratory modulating agent from the injectable hydrogel so as to form a chemotaxis gradient in the environment around the hydrogel; and
applying a first external stimulus to the injectable hydrogel and/or body cavity of the subject, wherein the first external stimulus effective to stimulate migration of one or more cells within the subject to the injected injectable hydrogel; and
capturing and/or retaining one or more of the one or more cells within the hydrogel for a period of time.

31. The method of claim 30, wherein the one or more cells are selected from: cancer cells, cancer stem cells, circulating cancer cells, residual cancer cells, tumor microenvironment cells, immune cells, tumor initiating cells, cancer-associated fibroblasts, or any combination thereof.

32. The method of claim 30, wherein the method further comprises injecting, into a body cavity of a subject,
a. an injectable hydrogel of any of claims 1-29, or
b. one or more reagents capable of forming an injectable hydrogel of any of claims 1-29 so as to form an injected injectable hydrogel within the body cavity after injection.

31. The method of claim 30, further comprising exposing the injected injectable hydrogel to a second external stimulus to the hydrogel after a period of time sufficient to capture and/or retain one or more cells in the injectable hydrogel.

32. The method of claim 33, wherein the second external stimulus is capable of modifying, modulating, inhibiting growth of, and/or killing one or more cells captured and/or retained in the injectable hydrogel.

33. The method of claim 34, wherein the second external stimulus is an energy and wherein the energy is an electric energy, a light energy, a magnetic energy, a thermal energy, an acoustic energy, a chemical energy, a biochemical energy, a radiation energy, or any combination thereof.

34. The method of claim 35, wherein second the external stimulus is an acoustic energy or an electric energy.

35. The method of claim 36, wherein the acoustic energy is ultrasound.

36. The method of claim 36, wherein the electric energy is delivered by one or more probes, wherein
a. a cathode probe or cathode end of a probe is positioned in, adjacent to, or in proximity to the injectable hydrogel; or
b. an anode probe or anode end of a probe is positioned in, adjacent to, or in proximity to the injectable hydrogel.

37. The method of claim 38, wherein the one or more probes or other energy sources capable of delivering the second external stimulus are placed in operable proximity to the injected injectable hydrogel.

38. The method of claim 2, wherein one or more of the one or more cells originates from the body cavity microenvironment.

39. The method of claim 33, wherein the second external stimulus is capable of
a. damaging one or more cells attracted to, in immediate proximity of, contained in the injected injectable hydrogel, or any combination thereof;
b. killing or ablating one or more cells attracted to, in immediate proximity of, contained in the injected injectable hydrogel; or any combination thereof;
c. modifying the one or more cells attracted to, in immediate proximity of, contained in the injected injectable hydrogel, or any combination thereof; or
d. any combination thereof.

40. The method of claim 30, wherein the body cavity is a surgical cavity.

41. The method of claim 42, wherein the surgical cavity is formed from removing a tumor from the body of the subject.

42. The method of claim 42, wherein the body cavity is in the brain of the subject.

43. The method of claim 44, wherein the tumor is a glioma.

44. A method of treating a cancer in a subject, comprising performing the method as in any one of claims 30-45.

45. The method of claim 46, wherein the cancer is a glioblastoma.

46. The method of claim 47, wherein the glioblastoma is glioblastoma multiforme.

47. The method of claim 48, further comprising imaging the injected injectable hydrogel after injecting into a subject.

48. The method of claim 46, further comprising releasing one or more agents optionally included in the injectable hydrogel over a period of time.

49. The method of claim 46, further comprising removing the injected hydrogel after one or more cells are collected therein.

50. The method of claim 46, further comprising delivering to the subject one or more chemotherapeutics, therapeutic radiation, or both.
